(19) United States
(12) Patent Application Publication
(10) Pub. No.: US 2022/0093002 A1
(43) Pub. Date: Mar. 24, 2022

(54) DIGITAL APPARATUS AND APPLICATION FOR CANCER CACHEXIA THERAPY AND METHODS OF USE THEREOF

(71) Applicant: S-Alpha Therapeutics, Seoul (KR)

(72) Inventor: Seung Eun Choi, Seoul (KR)

(73) Assignee: S-Alpha Therapeutics, Seoul (KR)

(21) Appl. No.: 17/540,213

(22) Filed: Dec. 1, 2021

Related U.S. Application Data
(63) Continuation-in-part of application No. PCT/KR2021/006565, filed on May 26, 2021.
(60) Provisional application No. 63/029,997, filed on May 26, 2020.

(51) Int. Cl. G09B 19/00; A61N 1/36; A61B 5/0205; A61B 5/024; A61B 5/00; A61B 5/11; A61B 5/1172; G16H 10/60; G16H 20/30; G09B 5/02

(52) U.S. Cl. CPC G09B 19/003; A61B 2562/0204; A61B 5/02055; A61B 5/024; A61B 5/0077; A61B 5/1112; A61B 5/1172; A61B 5/1118; A61B 5/4806; A61B 5/4833; G16H 10/60; G16H 20/30; G09B 5/02; A61B 2562/0219; A61B 2562/0223; A61B 2560/0242; A61N 1/36053

(57) ABSTRACT

Systems and methods for cancer cachexia therapy are provided. A system may include a digital apparatus, which may include a digital instruction generation unit configured to generate digital therapeutic modules for treating cancer cachexia, generate specified digital instructions based on the digital therapeutic modules and provide the digital instructions to a first user, and an outcome collection unit configured to collect the first user's execution outcomes of the digital instructions.

### BACKGROUND

Cancer cachexia is characterized by an ongoing loss of skeletal muscle mass (with or without loss of fat mass) that cannot be fully reversed by conventional nutrition. Current therapies for cachexia include medication aimed at retarding or halting progression of the disorder. Treatments include, for example, orexigenic agents (i.e., appetite stimulants), corticosteroids, cannabinoids, serotonin antagonists, prokinetic agents, androgens and anabolic agents, anticytokine agents, non-steroidal anti-inflammatory drugs, and regulators of circadian rhythm, with most therapies directed to treating the underlying or associated condition (e.g., cancer). However, such treatment (e.g., using small molecules, biologics, etc.) is often compromised by the patient's inability to tolerate the treatment due to their cachexia. Thus, there is a need in the art for improved treatments (e.g., digital therapeutics) for wasting disorders, such as cachexia.

### SUMMARY OF THE DISCLOSURE

The present disclosure relates to digital therapeutics (hereinafter referred to as DTx) intended for cancer cachexia therapy. The present disclosure also relates to systems that integrate digital therapeutics with one or both of a healthcare provider portal and an administrative portal to treat cancer cachexia in a patient. Some embodiments of the present disclosure may comprise deducing a mechanism of action (hereinafter referred to as MOA) in cancer cachexia, and establishing a therapeutic hypothesis and a digital therapeutic hypothesis for inhibiting progression of cancer cachexia, and treating the cancer cachexia based on these findings.

In some aspects, the present disclosure provides a method of treating cancer cachexia in a subject in need thereof, the method comprising: providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow, wherein the electronic device (i) comprises a sensor sensing adherence by the subject to the first instructions of the one or more first modules, (ii) transmits adherence information, based on the adherence, to a server, and (iii) receives one or more second instructions from the server based on the adherence information; and providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions.

In some aspects, the present disclosure provides a computing system for treating cancer cachexia in a subject in need thereof, comprising a display configured to provide, to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; a sensor configured to sense adherence by the subject to the instructions of the one or more first modules; a transmitter configured to transmit adherence information, based on the adherence, to a server; and a receiver configured to receive, from the server, one or more second instructions based on the adherence information, wherein the display is further configured to provide, to the subject, one or more second modules selected from the group consisting of a voluntary skeletal muscle exercise module, a vagal nerve stimulation module, an aerobic exercise module, and a relaxation module, each of the one or more second modules comprising the one or more second instructions.

In some aspects, the present disclosure provides a non-transitory computer readable medium having stored thereon software instructions for treating cancer cachexia in a subject in need thereof that, when executed by a processor, cause the processor to: display, by an electronic device to the subject, one or more first modules selected from the group consisting of a voluntary skeletal muscle exercise module, a vagal nerve stimulation module, an aerobic exercise module, and a relaxation module, each of the one or more first modules comprising instructions for the subject to follow; sense, by a sensor in the electronic device, adherence by the subject to the instructions of the one or more first modules; transmit, by the electronic device, adherence information, based on the adherence, to a server; receive, from the server, one or more second instructions based on the adherence information; and display, to the subject, one or more second modules selected from the group consisting of a voluntary skeletal muscle exercise module, a vagal nerve stimulation module, an aerobic exercise module, and a relaxation module, the one or more second modules comprising the one or more second instructions.

In some aspects, the present disclosure provides a system for treating cancer cachexia in a subject, comprising: a digital apparatus configured to execute a digital application comprising one or more first modules, for treating cancer cachexia in a subject, wherein the digital apparatus comprises a sensor for sensing adherence by the subject to a first set of instructions of the one or more first modules; a healthcare provider portal configured to provide one or more options to a healthcare provider to perform one or more tasks to prescribe treatment for the cancer cachexia in the subject based on information received from the digital application; and an administrative portal configured to provide one or more options to an administrator of the system to perform one or more tasks to manage access to the system by the healthcare provider.

In some embodiments, the digital application for treating cancer cachexia instructs a processor of the digital apparatus to execute operations comprising: generating digital therapeutic modules for treating cancer cachexia based on a mechanism of action in and a therapeutic hypothesis for the cancer cachexia. In some embodiments, the generating of the digital therapeutic modules comprises generating the digital therapeutic modules based on biochemical factors related to the cancer cachexia. In some embodiments, the one or more first modules comprise the voluntary skeletal muscle exercise module, and the voluntary skeletal muscle exercise module comprises one or more first instructions to increase MGF/IGF-1 secretion in the subject. In some embodiments, the one or more first instructions comprise instructions for aerobic, resistance, and/or concurrent exercise. In some embodiments, the one or more first modules comprise the vagal nerve stimulation module, and the vagal nerve stimulation module comprises one or more first instructions to reduce inflammation in the subject. In some embodiments, the one or more first modules comprise the vagal nerve stimulation module, and the vagal nerve stimulation module comprises at least one instruction selected from the group consisting of sense stimulation instructions for sight, sound, touch, taste, and smell. In some embodiments, the vagal nerve stimulation module comprises one or more sense stimulation instructions for sight, and the one or more sense stimulation instructions for sight include one or more instructions to view one or more figures (define in spec to include specific photos) to stimulate autonomic nervous system. In some embodiments, the electronic device receives and displays the figures. In some embodiments, the vagal nerve stimulation module comprises one or more sense stimulation instructions for sound, and the one or more sense stimulation instructions for sound include one or more instructions to hear one or more sounds to cause horror or relaxation. In some embodiments, the electronic device receives and plays the sounds. In some embodiments, the vagal nerve stimulation module comprises one or more sense stimulation instructions for touch, and the one or more sense stimulation instructions for touch include one or more instructions for abdominal breathing, controlling rate of breathing, cold massage, coughing, and skin massage. In some embodiments, the vagal nerve stimulation module comprises one or more sense stimulation instructions for touch, and the one or more sense stimulation instructions for touch include one or more instructions for abdominal breathing. In some embodiments, the vagal nerve stimulation module comprises one or more sense stimulation instructions for taste, and the one or more sense stimulation instructions for taste include one or more instructions to eat food to stimulate digestive glands in the subject. In some embodiments, the electronic device receives and displays information related to the food. In some embodiments, the vagal nerve stimulation module comprises one or more sense stimulation instructions for smell, and the one or more sense stimulation instructions for smell include one or more instructions to stimulate digestive glands and/or to relax. In some embodiments, the electronic device is configured to release a scent for aroma therapy. In some embodiments, the one or more first modules comprise the aerobic exercise module, and the aerobic exercise module comprises one or more first instructions to increase adiponectin secretion in the subject. In some embodiments, the one or more first instructions comprise one or more instructions for walking, biking, aerobic dance and/or swimming. In some embodiments, the one or more first modules comprise the relaxation module, and the relaxation module comprises one or more first instructions to increase adiponectin secretion in the subject. In some embodiments, the one or more first instructions comprise one or more meditation instructions. In some embodiments, said one or more first instructions comprise one or more sound instructions to hear relaxing sound. In some embodiments, the device receives and plays the relaxing sound. In some embodiments, the subject is an early cancer patient. In some embodiments, the subject has a cancer mass having a diameter of 3 cm or less. In some embodiments, the subject is a late cancer patient, and the system excludes providing a voluntary skeletal muscle exercise module. In some embodiments, the subject has a cancer mass having a diameter of more than 3 cm, and the system excludes providing a voluntary skeletal muscle exercise module. In some embodiments, the subject has moderate muscle atrophy, and the one or more first modules consists of the voluntary skeletal muscle exercise module, the vagal nerve stimulation module, the aerobic exercise module, and the relaxation module. In some embodiments, the subject has moderate muscle atrophy, and the one or more first modules consists of the vagal nerve stimulation module, the aerobic exercise module, and the relaxation module. In some embodiments, the subject has moderate muscle atrophy (in spec, define the moderate muscle atrophy as still being able to walk). In some embodiments, the subject has severe muscle atrophy (in spec, define the severe muscle atrophy as not being able to walk), and the system excludes providing a voluntary skeletal muscle exercise module and further excludes providing an aerobic exercise module. In some embodiments, the one or more first modules consists of the vagal nerve stimulation module and the relaxation module. In some embodiments, the digital application transmits data to a server, and wherein the server receives the one or more second instructions from an external reviewer. In some embodiments, the external reviewer comprises a health professional. In some embodiments, the external reviewer comprises an artificial intelligence (AI). In some embodiments, the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer. In some embodiments, the sensor comprises a touch sensor, and the subject provides the adherence information to the electronic device using the touch sensor. In some embodiments, the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI). In some embodiments, the digital application further comprises a push alarm for one or more of reminding the subject complete a digital therapeutic module. In some embodiments, the digital apparatus comprises a digital instruction generation unit configured to generate digital therapeutic modules for treating cancer cachexia, generate digital instructions based on the digital therapeutic modules, and provide the digital instructions to the subject; In some embodiments, an outcome collection unit configured to collect the subject's execution outcomes of the digital instructions. In some embodiments, the digital instruction generation unit generates the digital therapeutic modules based on biochemical factors related to the cancer cachexia onset. In some embodiments, the biochemical factors comprise insulin-like growth factor 1 (IGF1) and hypoxia-inducible factor 1 (HIF1). In some embodiments, the digital instruction generation unit generates the digital therapeutic modules based on the inputs from the healthcare provider. In some embodiments, the digital instruction generation unit generates the digital therapeutic modules based on information received from the subject. In some embodiments, the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus. In some embodiments, the digital instruction generation unit generates the digital therapeutic modules matching to imaginary parameters which correspond to the mechanism of action in and the therapeutic hypothesis for the cancer cachexia. In some embodiments, the imaginary parameters are deduced in relation to the subject's environment, behaviors, emotions, and cognition. In some embodiments, the outcome collection unit collects the execution outcomes of the digital instructions by monitoring the subject's adherence to the digital instructions or allowing the subject to directly input the subject's adherence to the digital instructions. In some embodiments, the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

### BRIEF DESCRIPTION OF THE DRAWINGS

The above and other objects, features and advantages of the present disclosure will become more apparent to those of ordinary skill in the art by describing in detail exemplary embodiments thereof with reference to the attached drawings, in which:

FIG. 1 illustrates muscle atrophy associated with cancer cachexia;

FIG. 2 illustrates various biochemical pathways and physiological symptoms associated with cancer cachexia, as well as applications of certain embodiments of the present disclosure and associated effects;

FIG. 3 is a block diagram showing an exemplary configuration of a digital apparatus for treating cancer cachexia according to one embodiment of the present disclosure;

FIG. 4 is a diagram showing exemplary input and output loops of a digital application for treating cancer cachexia according to one embodiment of the present disclosure;

FIG. 5 is a diagram showing an exemplary background factors supporting the digital apparatus and the application for treating cancer cachexia according to one embodiment of the present disclosure;

FIG. 6 depicts diagrams showing an exemplary method of assigning a patient-customized digital prescription using the digital apparatus and the application for treating cancer cachexia according to one embodiment of the present disclosure;

FIG. 7 is a flowchart illustrating exemplary operations in a digital application for treating cancer cachexia according to one embodiment of the present disclosure;

FIG. 8 is a diagram showing an exemplary hardware configuration of the digital apparatus for treating cancer cachexia according to one embodiment of the present disclosure;

FIG. 9 is a flow chart illustrating an exemplary system for treating cancer cachexia, the system comprising an administrative portal (e.g., Administrator's web), a healthcare provider portal (e.g., Doctor's web) and a digital apparatus configured to execute a digital application (e.g., an application or ‘app’) for treating cancer cachexia in a subject;

FIG. 10 is a flow chart illustrating an exemplary digital application usage flow of the present disclosure;

FIG. 11 is a flow chart illustrating an exemplary execution flow for a login verification during a splash process at the starting of a digital application of the present disclosure;

FIG. 12 is a diagram illustrating an exemplary patient portal structure of a digital application of the present disclosure;

FIG. 13 is a flow chart illustrating an exemplary patient portal of internal activity use flow of a digital application of the present disclosure;

FIG. 14 is a flow chart illustrating an exemplary patient portal structure of the present disclosure.

FIG. 15 is a flow chart illustrating an exemplary usage flow of a digital application of the present disclosure.

FIG. 16 is a flow chart illustrating an exemplary target heart rate calculation formula of a digital application of the present disclosure.

FIG. 17 is a diagram illustrating an exemplary scheduled exercise sessions of a digital application of the present disclosure.

FIG. 18 is a diagram illustrating an exemplary patient portal structure of a digital application of the present disclosure.

FIG. 19 is a flow chart illustrating an exemplary doctor portal structure and administrative portal structure of a digital application of the present disclosure.

FIG. 20 is a flow chart illustrating an exemplary execution flow for an administrative portal in a system of the present disclosure.

FIG. 21 is a diagram illustrating an exemplary usage flow of a digital application of the present disclosure.

FIG. 22 is a diagram illustrating an exemplary screen touch sensing configuration for the voluntary skeletal muscle exercise module.

FIG. 23 is a diagram illustrating an exemplary usage flow of a digital application of the present disclosure.

FIG. 24 is a diagram illustrating an exemplary head lifting sensing configuration for the voluntary skeletal muscle exercise module.

FIG. 25 is a diagram illustrating an exemplary head turning sensing configuration for the voluntary skeletal muscle exercise module.

FIG. 26 is a flow chart illustrating an exemplary execution flow for the head turning instructions for the voluntary muscle exercise module.

FIG. 27 is a diagram illustrating an exemplary usage flow of a digital application of the present disclosure.

FIG. 28 is a flow chart illustrating an exemplary execution flow for the hand gripping instructions for the voluntary muscle exercise module.

FIG. 29 is a diagram illustrating an exemplary usage flow of a digital application of the present disclosure.

FIG. 30 is a diagram illustrating an exemplary arm shaking configuration for the voluntary skeletal muscle exercise module.

FIG. 31 is a diagram illustrating an exemplary leg lifting configuration for the voluntary skeletal muscle exercise module.

FIG. 32 is a flow chart illustrating an exemplary execution flow for the leg lifting instructions for the voluntary muscle exercise module.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### DETAILED DESCRIPTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may mean a range of +20%, +10%, or +5% of a given numeric value.

As used herein, “muscle atrophy” can refer to a disease in which muscles of the body (e.g., legs and arms) are gradually atrophied, sometimes symmetrically. Muscle atrophy may accompany the occurrence of cancer, aging, renal diseases, genetic diseases, and various chronic diseases.

As used herein, the term “cancer cachexia” can refer to a condition characterized by an ongoing loss of skeletal muscle mass (with or without loss of fat mass) that cannot be fully reversed by conventional nutrition.

Overview

With reference to the appended drawings, exemplary embodiments of the present disclosure will be described in detail below. To aid in understanding the present disclosure, like numbers refer to like elements throughout the description of the figures, and the description of the same elements will be not reiterated.

Development of new drugs starts with confirming a medial demand in situ, proposing a mechanism of action based on the expert reviews and meta-analysis on the corresponding disease, and deducing a therapeutic hypothesis based on the expert reviews and the meta-analysis. Also, after a library of drugs whose therapeutic effects are expected is prepared based on the therapeutic hypothesis, a candidate material is found through screening, and the corresponding candidate material is subjected to optimization and preclinical trials to check its effectiveness and safety from a preclinical stage, thereby deciding the candidate material as a final candidate drug. To mass-produce the corresponding candidate drug, a CMC (chemistry, manufacturing, and control) process is also established, a clinical trial is carried out on the corresponding candidate drug to verify a mechanism of action and a therapeutic hypothesis of the candidate drug, thereby ensuring the clinical effectiveness and safety of the candidate drug.

From the point of view of this patent, drug targeting and signaling, which fall upstream of the development of new drugs, have many uncertainties. In many cases, because the drug targeting and signaling take a methodology of putting together the outcomes, which have been reported in the art, and interpreting the outcomes, it may be difficult to guarantee the novelty of disclosure. On the contrary, the disclosure of drugs capable of regulating the drug targeting and signaling to treat a disease requires the highest level of creativity except for the field of some antibody or nucleic acid (DNA, RNA) therapeutics in spite of the development of research methodology for research and development of numerous new drugs. As a result, the molecular structures of the drugs are the most critical factors that constitute the most potent substance patent in the field of new drugs.

Unlike the drugs whose rights are strongly protected through this substance patent, digital therapeutics are basically realized using software. Due to the nature of the digital therapeutics, the rational design of digital therapeutics against the corresponding disease, and the software realization of the digital therapeutics based on the rational design may be considered to be an improved process of disclosure to be protected as a patent when considering the clinical verification and approval processes as the therapeutics.

That is, the core of the digital therapeutics as in the present disclosure depends on the rational design of digital therapeutics suitable for treatment of the corresponding disease, and the development of specific software capable of clinically verifying the digital therapeutics based on the rational design. Hereinafter, a digital apparatus and an application for treating cancer cachexia according to the present disclosure realized in this aspect will be described in detail.

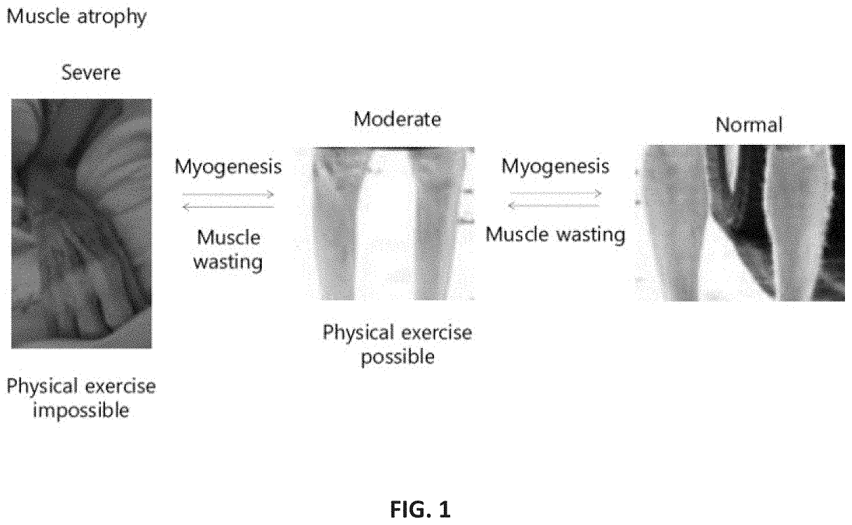

FIG. 1 illustrates the progression of muscle atrophy, which is one aspect of cancer cachexia (the other aspect being cancer). As used herein, “muscle atrophy” can refer to a disease in which muscles of the body (e.g., legs and arms) are gradually atrophied, sometimes symmetrically. Muscle atrophy may accompany the occurrence of cancer, aging, renal diseases, genetic diseases, and various chronic diseases. Muscle atrophy is represented by amyotrophic lateral sclerosis (Lou Gehrig's disease), spinal progressive muscular atrophy, etc. A normal or healthy individual (far right) is capable of exercise. Muscle wasting or atrophy can lead to moderate muscular atrophy, where physical exercise is still possible. Moderate muscle atrophy can be characterized by reduced motor function, wherein the patient can still walk, although not as well as a healthy individual (e.g., a patient with moderate muscle atrophy can have an abnormal gait, slouch, and/or have a rigid posture, etc.). Further muscle wasting can lead to severe muscle atrophy, where physical exercise is no longer possible. Severe muscle atrophy can be characterized by reduced or complete loss of motor function, such as an inability to walk. Generally, the stage of muscle atrophy can be determined or classified by muscle volume and/or athletic ability. Likewise, myogenesis can restore muscle function. The methods and systems of the present disclosure can promote myogenesis in patients suffering from severe and moderate muscle atrophy to restore normal muscle function. For patients suffering from muscle atrophy, methods and system of the present disclosure can be helpful in promoting myogenesis such that the muscle atrophy is reduced, and the patient is able to tolerate a cancer treatment that may not otherwise be possible, for example, in a patient suffering from severe muscle atrophy.

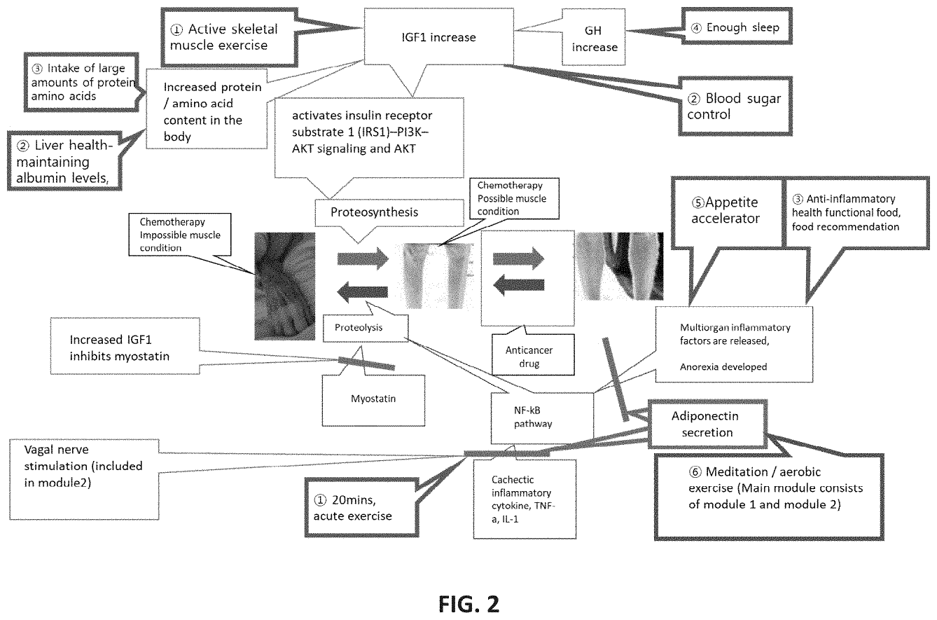

FIG. 2 illustrates various exemplary biochemical pathways and physiological symptoms associated with cancer cachexia, as well as exemplary applications of certain embodiments of the present disclosure and associated effects. Cachexia can be characterized by an acquired, accelerated loss of muscle caused by an underlying disease. When cachexia is seen in a patient with cancer, the condition may be referred to as “cancer cachexia”. Cancer cachexia affects the majority of patients with advanced cancer and is associated with a reduction in treatment tolerance, response to therapy, quality of life and duration of survival. Cancer cachexia is a multifactorial syndrome characterized by an ongoing loss of skeletal muscle mass, with or without loss of fat mass, which cannot be fully reversed by conventional nutritional support and leads to progressive functional impairment. Skeletal muscle loss may be the most significant event in cancer cachexia.

As mentioned above, one aim of the methods and systems of the present disclosure is to at least partially reduce muscle atrophy such that a patient is able to tolerate cancer treatment. Muscle atrophy can be caused by increased myostatin levels, which promote proteolysis. In one aspect, methods and systems of the present disclosure increase insulin-like growth factor 1 (IGF1) and/or a splice variant of IGF-1 called mechano growth factor (MGF) levels (IGF1/MGF level), for example, using voluntary skeletal muscle exercise modules and/or digital instructions for a patient's diet, which can (i) inhibit myostatin and prevent muscular atrophy associated with proteolysis, and/or (ii) activate insulin receptor substrate 1 (IRS1)-PI3K-AKT signaling and AKT to promote proteosynthesis. Muscle atrophy can also result from activation of the NF-κB pathway (e.g., via release of multiorgan inflammatory factors and/or cachetic inflammatory cytokines TNF-α/IL-1), which promote proteolysis. In one aspect, methods and systems of the present disclosure inhibit activation of the NF-κB pathway by promoting adiponectin secretion, for example, through meditation and/or aerobic exercise. In another aspect, methods and systems of the present disclosure inhibit activation of the NF-κB pathway by inhibiting cachetic inflammatory cytokines TNF-α/IL-1 through vagal nerve stimulation.

In addition to cachexia, cancer is another aspect of cancer cachexia. For early stage cancer patients, stimulating IGF1 secretion, for example, by physical exercise, may help treat cancer cachexia. In patients with terminal cancer, however, the above strategy may have side effects that promote HIF1 and accelerate cancer progression. Thus, for a late stage cancer, cancer cachexia therapy may exclude treatments to stimulate IGF1 secretion, such as a physical exercise. Accordingly, the prescription of module assigned to each patient can be different according to the progress of cancer. Thus, the early external activity for the early state cancer patients may be different from the late external activity for the late stage cancer patients as described herein.

In some embodiments, the late external activity includes touching a screen with a finger or a heel, and deep breathing. The touching may be monitored by a touch sensor, acceleration sensor, and/or gyro sensor. In additional embodiments, the late external activity includes lifting a head while lying down, and raising a hand or leg while lying down, which may be monitored by acceleration sensor, gyro sensor, or a touch sensor. In further embodiments, the late external activity includes grabbing phone while lying down, turning a head while lying down. The grabbing may be monitored by a touch sensor or a gesture recognition. The head turning may be monitored by a face recognition.

A digital apparatus and an application for inhibiting progression of and treating cancer cachexia according to the present disclosure will be described below.

Generally speaking, disease therapy is carried out by analyzing a certain disease in terms of pathophysiological functions and dispositions in order to determine a start point, a progression point, and an end point for the disease. Also, an indication of the disease is defined by characterization of the corresponding disease and statistical analysis of the disease. Also, patient's physiological factors, especially biochemical factors, which correspond to the verified indications, are analyzed, and the patient's biochemical factors are restricted a narrow extent associated with the disease to deduce a mechanism of action.

Next, a therapeutic hypothesis, in which the corresponding disease is treated by controlling actions and environments directly associated with regulation of the corresponding biochemical factors associated with the disease, is deduced. To realize this therapeutic hypothesis into digital therapeutics, a digital therapeutic hypothesis for achieving a therapeutic effect through repeated digital instruction and execution, which are associated with the “control of patient's action/environment→regulation of biochemical factors, is proposed. The digital therapeutic hypothesis of the present disclosure is realized as a digital apparatus and an application is realized as a digital apparatus and an application configured to present changes in patient's actions, and patient's participation in the form of specific instructions and collect and analyze execution of the specific instructions.

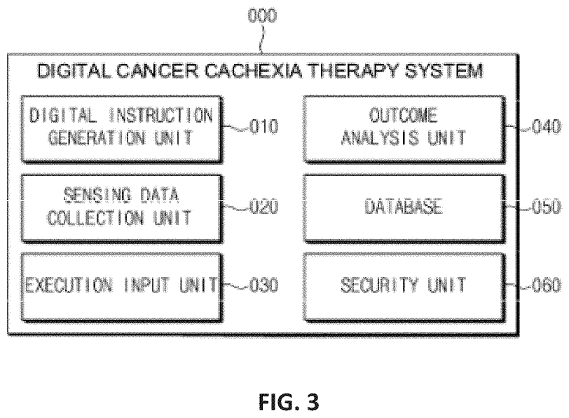

FIG. 3 is a block diagram showing a configuration of the digital apparatus for treating cancer cachexia according to one embodiment of the present disclosure. Referring to FIG. 3, a digital system 000 for treating cancer cachexia according to one embodiment of the present disclosure may include a digital instruction generation unit 010, a sensing data collection unit 020, an execution input unit 030, an outcome analysis unit 040, a database 050, and a security unit 060.

Based on the mechanism of action in and the therapeutic hypothesis and digital therapeutic hypothesis for cancer cachexia, a doctor (a second user) may prescribe digital therapeutics, which are realized in a digital apparatus and an application for treating cancer cachexia, for the corresponding patient. In this case, the digital instruction generation unit 010 is a device configured to provide a prescription of the digital therapeutics to a patient as a specific behavioral instruction that the patient may execute based on the interaction between the biochemical factors for cancer cachexia and the patient's behaviors. For example, the biochemical factors may include IGF1, HIF1, and the like, but the present disclosure is not limited thereto. For example, all types of biochemical factors that may cause cancer cachexia may be considered.

The digital instruction generation unit 010 may generate digital instructions based on the inputs from the doctor. In this case, the digital instruction generation unit 010 may generate digital instructions based on information collected by the doctor when diagnosing a patient. Also, the digital instruction generation unit 010 may generate digital instructions based on the information received from the patient. For example, the information received from the patient may include the patient's basal factors, medical information, and digital therapeutics literacy. In this case, the basal factors may include amount of the patient's activity, heart rates, sleep, meals (nutrition and calories), and the like. The medical information may include the patient's electronic medical record (EMR), family history, genetic vulnerability, genetic susceptibility, and the like. The digital therapeutics literacy may include the patient's accessibility and an acceptance posture to the digital therapy instructions and the apparatus, and the like.

The digital instruction generation unit 010 may reflect the mechanism of action in and the therapeutic hypothesis for cancer cachexia in order to utilize imaginary parameters and generate a digital module. In this case, the imaginary parameters may be deduced in term of the patient's behaviors.

The digital instruction generation unit 010 generates digital instructions particularly designed to allow a patient to have a therapeutic effect, and provides the instructions to the patient. For example, the digital instruction generation unit 010 may generate specific digital instructions in each of digital therapeutic modules.

The sensing data collection unit 020 and the execution input unit 030 may collect the patient's execution outcomes of the digital instructions provided at the digital instruction generation unit 010. Specifically, the sensing data collection unit 020 configured to sense the patient's adherence to the digital instructions and the execution input unit 030 configured to allow a patient to directly input the execution outcomes of the digital instructions are included, and thus serve to output the patient's execution outcomes of the digital instructions.

The outcome analysis unit 040 may collect the patient's behavior adherence or participation in predetermined periods and report the patient's behavior adherence or participation to external systems. Therefore, a doctor may continue to monitor an execution course of the digital instructions through the application even when a patient does not directly visit a hospital.

The database 050 may store the mechanism of action in cancer cachexia, the therapeutic hypothesis for cancer cachexia, the digital instructions provided to the user, and the user's execution outcome data. FIG. 3 shows that the database 050 is included in the digital apparatus 000 for treating cancer cachexia. However, the database 050 may be provided in an external server.

Meanwhile, a series of loops including inputting the digital instructions at the digital instruction generation unit 010, outputting the patient's execution outcomes of the digital instructions at the sensing data collection unit 020/execution input unit 030, and evaluating the execution outcomes at the outcome analysis unit 040 may be repeatedly executed several times. In this case, the digital instruction generation unit 010 may generate patient-customized digital instructions for this cycle by reflecting the patient's digital instructions provided in the previous cycle and output values, and the evaluation.

As described above, according to the digital therapy apparatus for inhibiting progression of and treating cancer cachexia according to the present disclosure, the cancer cachexia therapy whose reliability may be ensured is possible by deducing the mechanism of action in cancer cachexia and the therapeutic hypothesis and digital therapeutic hypothesis for cancer cachexia in consideration of the biochemical factors for cancer cachexia, presenting digital instructions for treating cancer cachexia based on the mechanism of action and the therapeutic hypotheses, and collecting and analyzing execution of specific instructions.

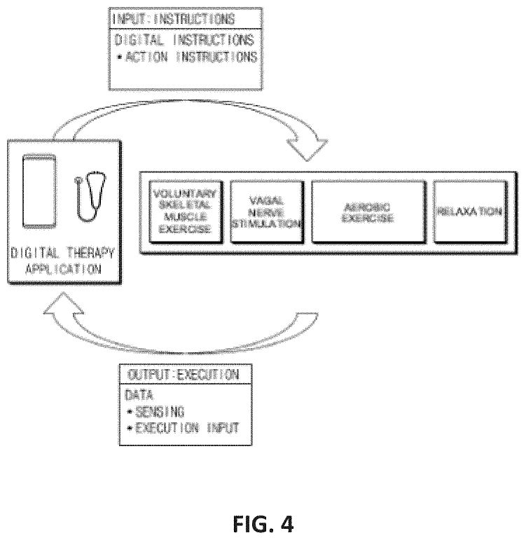

FIG. 4 is a diagram showing input and output loops of the digital application for treating cancer cachexia according to one embodiment of the present disclosure.

Referring to FIG. 4, the digital application for treating cancer cachexia according to one embodiment of the present disclosure may input the corresponding digital prescription for a patient in the form of instructions, and may output execution outcomes of the corresponding digital instructions.

The digital instructions provided to the patient may include specific action instructions for behaviors, and the like. As shown in FIG. 4, the digital instructions may include voluntary skeletal muscle exercise, vagal nerve stimulation, aerobic exercise, relaxation, and the like. However, the digital instructions are given by way of illustration only, and are not intended to be limiting to the digital instruction according to the present disclosure.

The patient's execution outcomes of the digital instructions consist of 1) log-in/log-out information for instructions and execution, 2) adherence information sensed as passive data such as voluntary skeletal muscle exercise, heart rates associated with the stress, a change in oxygen saturation, and the like, and 3) directly input information on the patient's execution outcomes.

The inhibition of the progression of and the treatment of cancer cachexia are shown to be achieved by repeatedly executing the aforementioned single feedback loop of FIG. 4 several times to regulate the biochemical factors.

Inhibitory and therapeutic effects on progression of the cancer cachexia may be more effectively achieved by gradual improvement of an instruction-execution cycle in the feedback loop, compared to the simply repeated instruction-execution cycle during the corresponding course of therapy.

For example, the digital instructions and the execution outcomes for the first cycle are given as input values and output values in a single loop, but new digital instructions may be generated by reflecting input values and output values generated in this loop using a feedback process of the loop to adjust the input for the next loop when the feedback loop is executed N times. This feedback loop may be repeated to deduce patient-customized digital instructions and maximize a therapeutic effect at the same time.

As such, in the digital apparatus and the application for treating cancer cachexia according to one embodiment of the present disclosure, the patient's digital instructions provided in the previous cycle (for example, a N−1^{st} cycle), and the data on instruction execution outcomes may be used to calculate the patient's digital instructions and execution outcomes in this cycle (for example, a N^{th} cycle). That is, the digital instructions in the next loop may be generated based on the patient's digital instructions and execution outcomes of the digital instructions calculated in the previous loop. In this case, various algorithms and statistical models may be used for the feedback process, when necessary.

As described above, in the digital apparatus and the application for treating cancer cachexia according to one embodiment of the present disclosure, it is possible to optimize the patient-customized digital instructions suitable for the patient through the rapid feedback loop.

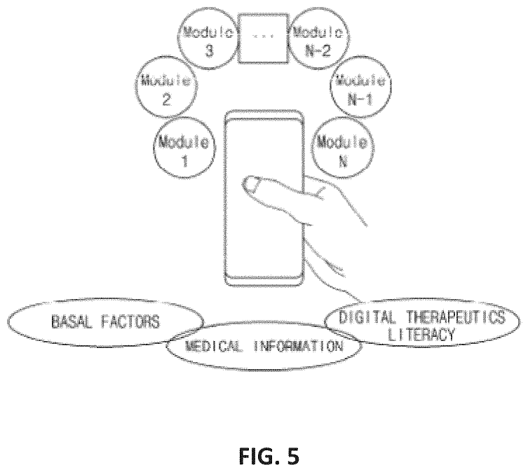

FIG. 5 is a diagram showing a background factors supporting the digital apparatus and the application for treating cancer cachexia according to one embodiment of the present disclosure

Referring to FIG. 5, the background factors may be considered together in the design of the modules in the digital apparatus and the application for treating cancer cachexia according to one embodiment of the present disclosure.

In this case, the background factors are elements necessary for correction of clinical trial outcomes during verification of the clinical effectiveness of digital cancer cachexia therapy according to the present disclosure. Specifically, in the background factors shown in FIG. 5, the basal factors may include activity, heart rates, sleep, meals (nutrition and calories), and the like, the medical information may include EMR, family history, genetic vulnerability, and susceptibility, and the like, which have been written when a patient visited a hospital, and the digital therapeutics literacy may include the patient's accessibility to the digital therapy instructions and the apparatus, and an acceptance posture.

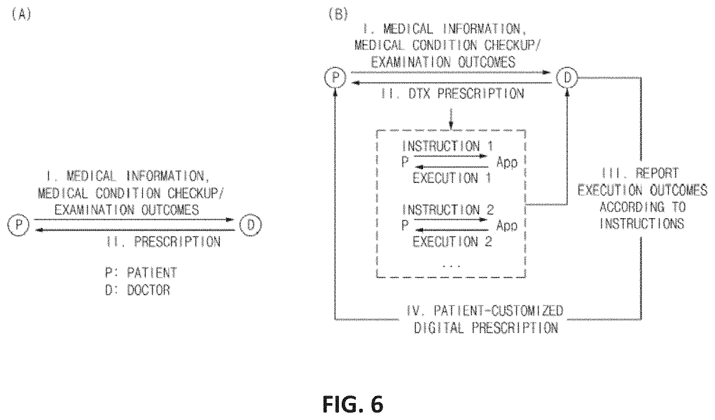

FIG. 6 is a diagram showing a method of assigning a patient-customized digital prescription using the digital apparatus and the application for treating cancer cachexia according to one embodiment of the present disclosure.

FIG. 6(A) show a prescription procedure for routine medical condition checkup of a patient by a doctor, and FIG. 6(B) show a method of allowing a doctor to assign a patient-customized digital prescription based on the analysis of a plurality of digital instructions and execution outcomes of the digital instructions.

In this way, when the digital apparatus and the application for treating cancer cachexia according to one embodiment of the present disclosure are used, the doctor may check the patient's instructions and execution outcomes for a given period and adjust the types of modules for treating cancer cachexia, and the instructions for each module in a patient-customized manner, as shown in FIG. 6(B).

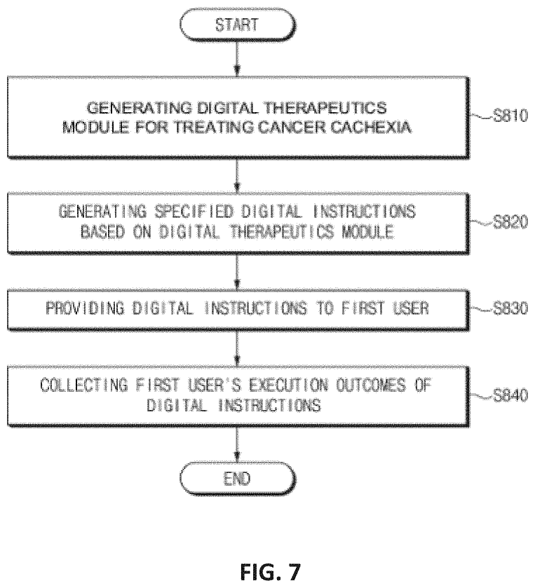

FIG. 7 is a flowchart illustrating operations in the digital application for treating cancer cachexia according to one embodiment of the present disclosure.

Referring to FIG. 7, the digital application for treating cancer cachexia according to one embodiment of the present disclosure may first generate a digital therapeutics module for treating cancer cachexia based on the mechanism of action in and the therapeutic hypothesis for cancer cachexia (S810). In this case, in S810, the digital therapeutics module may be generated based on the biochemical factors (for example, IGF1, HIF1, etc.) for cancer cachexia.

Meanwhile, in S810, the digital therapeutics module may be generated based on the inputs from the doctor. In this case, a digital therapeutics module may be generated based on the information collected by the doctor when diagnosing a patient, and the prescription outcomes recorded based on the information. Also, in S810, the digital therapeutics module may be generated based on the information (for example, basal factors, medical information, digital therapeutics literacy, etc.) received from the patient.

Next, in S820, specified digital instructions may be generated based on the digital therapeutics module. S820 may generate a digital therapeutics module by applying imaginary parameters about the patient's environments, behaviors, emotions, and cognition to the mechanism of action in and the therapeutic hypothesis for cancer cachexia.

In this case, the digital instructions may be generated for at least one of voluntary skeletal muscle exercise, vagal nerve stimulation, aerobic exercise, and relaxation modules. Then, the digital instructions may be provided to a patient (S830). In this case, the digital instructions may be provided in the form of digital instructions which are associated with behaviors and in which the patient's instruction adherence such as voluntary skeletal muscle exercise may be monitored using a sensor, or provided in the form of digital instructions in which a patient is allowed to directly input the execution outcomes.

After the patient executes the presented digital instructions, the patient's execution outcomes of the digital instructions may be collected (S840). In S840, the execution outcomes of the digital instructions may be collected by monitoring the patient's adherence to the digital instructions as described above, or allowing the patient to input the execution outcomes of the digital instructions.

In some embodiments, the patient or caregiver would input a sleep time, blood sugar level, food intake or meal information, appetite stimulant or other drug intake information, albumin levels, early external activity, late external activity, and/or internal activity information daily, and all or part of the information may be monitored by a doctor, for example, on his web.

Meanwhile, the digital application for treating cancer cachexia according to one embodiment of the present disclosure may repeatedly execute operations several times, wherein the operations include generating the digital instruction and collecting the patient's execution outcomes of the digital instructions. In this case, the generating of the digital instruction may include generating the patient's digital instructions for this cycle based on the patient's digital instructions provided in the previous cycle and the execution outcome data on the patient's collected digital instructions provided in the previous cycle.

As described above, according to the digital application for treating cancer cachexia according to one embodiment of the present disclosure, the reliability of the inhibition of progression of and treatment of cancer cachexia may be ensured by deducing the mechanism of action in and the therapeutic hypothesis for cancer cachexia in consideration of the biochemical factors for cancer cachexia, presenting the digital instructions to a patient based on the mechanism of action in and the therapeutic hypothesis for cancer cachexia, and collecting and analyzing the outcomes of the digital instructions.

Although the digital apparatus and the application for treating cancer cachexia according to one embodiment of the present disclosure have been described in terms of cancer cachexia therapy, the present disclosure is not limited thereto. For the other diseases other than the cancer cachexia, the digital therapy may be executed substantially in the same manner as described above.

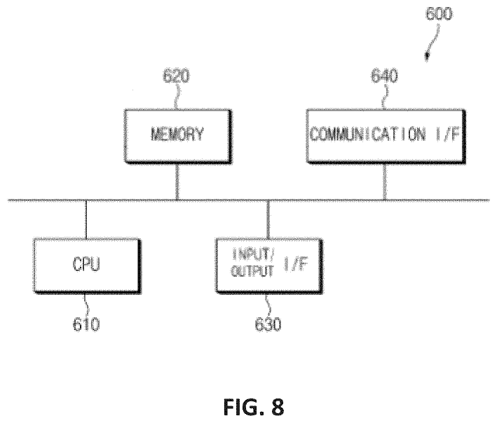

FIG. 8 is a diagram showing a hardware configuration of the digital apparatus for treating cancer cachexia according to one embodiment of the present disclosure.

Referring to FIG. 8, hardware 600 of the digital apparatus for treating cancer cachexia according to one embodiment of the present disclosure may include a CPU 610, a memory 620, an input/output I/F 630, and a communication I/F 640.

The CPU 610 may be a processor configured to execute a digital program for treating cancer cachexia stored in the memory 620, process various data for treating digital cancer cachexia and execute functions associated with the digital cancer cachexia therapy. That is, the CPU 610 may act to execute functions for each of the configurations shown in FIG. 3 by executing the digital program for treating cancer cachexia stored in the memory 620.

The memory 620 may have a digital program for treating cancer cachexia stored therein. Also, the memory 620 may include the data used for the digital cancer cachexia therapy included in the aforementioned database 050, for example, the patient's digital instructions and instruction execution outcomes, the patient's medical information, and the like.

A plurality of such memories 620 may be provided, when necessary. The memory 620 may be a volatile memory or a non-volatile memory. When the memory 620 is a volatile memory, RAM, DRAM, SRAM, and the like may be used as the memory 620. When the memory 620 is a non-volatile memory, ROM, PROM, EAROM, EPROM, EEPROM, a flash memory, and the like may be used as the memory 620. Examples of the memories 620 as listed above are given by way of illustration only, and are not intended to limit the present disclosure.

The input/output I/F 630 may provide an interface in which input apparatuses (not shown) such as a keyboard, a mouse, a touch panel, and the like, and output apparatuses such as a display (not shown), and the like may transmit and receive data (e.g., wirelessly or by hardline) to the CPU 610.

The communication I/F 640 is configured to transmit and receive various types of data to/from a server, and may be one of various apparatuses capable of supporting wire or wireless communication. For example, the types of data on the aforementioned digital behavior-based therapy may be received from a separately available external server through the communication I/F 640.

As described above, the computer program according to one embodiment of the present disclosure may be recorded in the memory 620 and processed at the CPU 610, for example, so that the computer program may be realized as a module configured to execute each of functional blocks shown in FIG. 3.

According to the digital apparatus and the application for treating, ameliorating, or preventing cancer cachexia according to the present disclosure, a reliable digital apparatus and application capable of inhibiting progression of and treating cancer cachexia may be provided by deducing a mechanism of action in cancer cachexia and a therapeutic hypothesis and a digital therapeutic hypothesis for cancer cachexia in consideration of biochemical factors for progression of cancer cachexia, presenting digital instructions to a patient, and collecting and analyzing execution outcomes of the digital instructions.

In certain embodiments, the present disclosure provides a method of treating, ameliorating, or preventing cancer cachexia in a subject in need thereof, the method comprising providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow, wherein the electronic device (i) comprises a sensor sensing adherence by the subject to the first instructions of the one or more first modules, (ii) transmits adherence information, based on the adherence, to a server, and (iii) receives one or more second instructions from the server based on the adherence information; and providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions.

In certain embodiments, the subject is diagnosed with cancer. In certain embodiments, the subject has cancer, and the subject is suffering from cancer cachexia. The subject may be a cancer patient, a patient at risk for cancer, or a patient with a family or personal history of cancer. In some cases, the patient is in a particular stage of cancer treatment. In certain embodiments, the subject has cancer, and the cancer is an early stage cancer. In certain embodiments, a subject with early stage cancer is a subject having a cancerous mass that is less than or equal to 0.1 centimeter (cm), less than or equal 0.5 cm, less than or equal 1 cm, less than or equal 2 cm, less than or equal 3 cm, less than or equal 4 cm, or less than or equal 5 cm in diameter. In certain embodiments, the subject has cancer, and the cancer is a late stage cancer. In certain embodiments, a subject with late stage cancer is a subject having a cancerous mass that is greater than or equal to 0.1 centimeter (cm), greater than or equal 0.5 cm, greater than or equal 1 cm, greater than or equal 2 cm, greater than or equal 3 cm, greater than or equal 4 cm, or greater than or equal 5 cm in diameter. Generally, the stage of cancer in a patient can be determined or classified based on HIF-1α expression levels (e.g., in the blood), hypoxia, and/or size of a cancerous mass in the subject.

Cancer patients may have any type of cancer. Examples of cancer can include, but are not limited to, adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, Kaposi sarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, or vulvar cancer. The term ‘lymphoma’ may refer to any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). The term ‘leukemia’ may refer to any type of leukemia including acute leukemia or chronic leukemia. Types of leukemia include acute myeloid leukemia, chronic myeloid leukemia, acute lymphocytic leukemia, acute undifferentiated leukemia, or chronic lymphocytic leukemia. In some cases, the cancer patient does not have a particular type of cancer. For example, in some instances, the patient may have a cancer that is not breast cancer.

Examples of cancer can include cancers that cause solid tumors as well as cancers that do not cause solid tumors. Furthermore, any of the cancers mentioned herein may be a primary cancer (e.g., a cancer that is named after the part of the body where it first started to grow) or a secondary or metastatic cancer (e.g., a cancer that has originated from another part of the body).

In some embodiments, the subject is at risk for cancer, and may be at risk because of a particular condition such as a pre-cancerous condition. Pre-cancerous conditions include but are not limited to actinic keratosis, Barrett's esophagus, atrophic gastritis, ductal carcinoma in situ, dyskeratosis congenita, sideropenic dysphagia, lichen planus, oral submucous fibrosis, solar elastosis, cervical dysplasia, leukoplakia, and erythroplakia). In some cases, a patient may be at risk of cancer because of cell or tissue dysplasia (e.g., an abnormal change in cell number, abnormal change in cell shape, abnormal change in cell size, or abnormal change in cell pigmentation).

In certain embodiments, the one or more first modules comprise the voluntary skeletal muscle exercise module. In certain embodiments, the voluntary skeletal muscle exercise module comprises one or more first instructions to increase MGF/IGF-1 secretion in the subject.

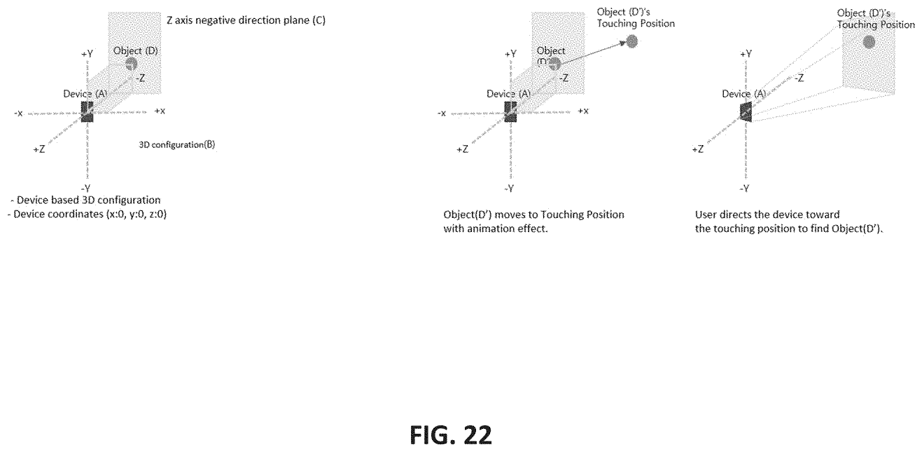

In certain embodiments, wherein the voluntary skeletal muscle exercise module comprises a screen touching exercise instruction to touch a randomly generated displayed animation on the electronic device. FIG. 22 is a diagram illustrating an exemplary screen touch sensing configuration for the voluntary skeletal muscle exercise module. The screen touch sensing configuration may be based on the electronic device's camera direction when running the digital application. The screen touch sensing configuration may be separated into the X-axis (left and right) and the Y-axis (up and down) (FIG. 22). In certain embodiments, the electronic device randomly generates a displayed animation of one object on the Z axis negative direction plane; When one object is touched, the selected object is removed, and a new object is created on the Z axis negative direction plane; with animation effects, the new object may appear on the screen of the electronic device for a brief period of time and moves to a new touching position; the electronic device may instruct the subject to move the device to a new location to find the new object and repeat the screen touching exercise. In certain embodiments, the screen touch sensing configuration may calculate the distance traveled by the device and record the data in the doctor portal and administrative portal (FIG. 22).

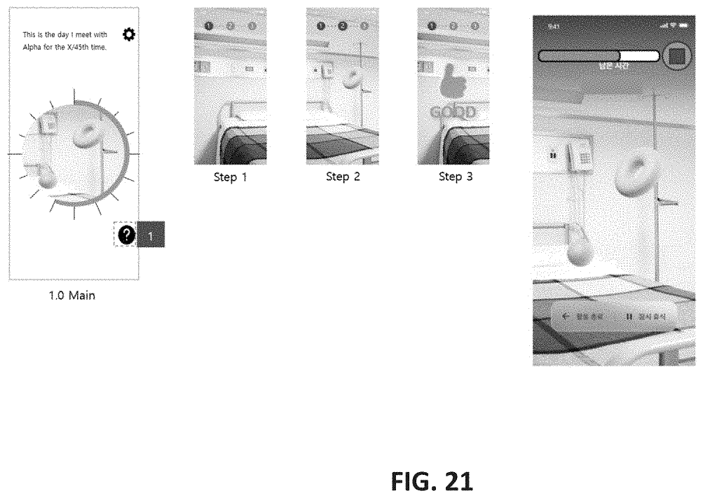

FIG. 21 is a diagram illustrating an exemplary usage flow of a digital application of the screen touching exercise of the present disclosure. At the start of the digital application, the subject may be instructed to perform screen touch exercises as an exemplary exercise of the voluntary skeletal muscle exercise modules. In some embodiments, the digital application may instruct the subject to pick up the electronic device and move the camera on the electronic device to find an animated object; when the animated object appears on the screen of the electronic device, the subject may be instructed to touch the animated object; When the subject touches the animated object, the tutorial ends. The digital application may instruct the subject to click the activity start button and activate a timer that measures the progress of the exercise module and the remaining time of the exercise module. The digital application may create coordinates dependent on the animated objects and their positions on the plane of the screen. The plane of the screen may be divided into four equal parts with maybe 1, 2, 3, or 4 animated objects displayed on different parts of the screen. The digital application may instruct the subject to touch the animated objects and repeat the exercise until the timer reaches 0.

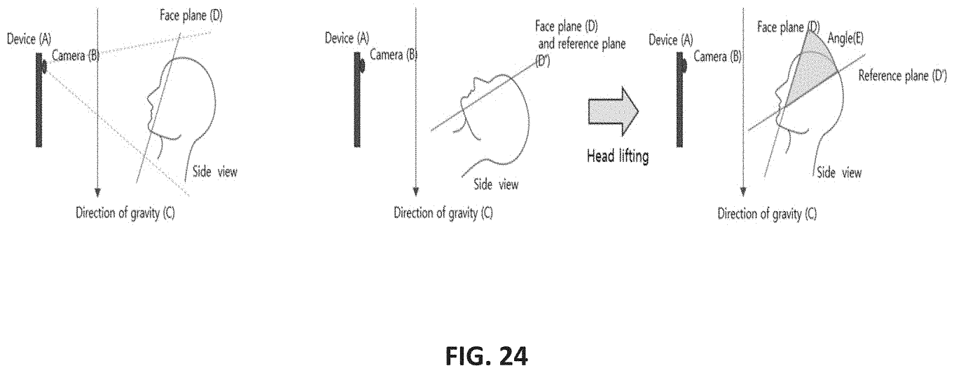

In certain embodiments, wherein the voluntary skeletal muscle exercise module comprises a head lifting exercise instruction to lift and position a head of the subject at a predetermined angle from the reference plane. FIG. 24 is a diagram illustrating an exemplary head lifting sensing configuration for the voluntary skeletal muscle exercise module. In certain embodiments, the electronic device recognizes the subject's face through the camera of the electronic device; The electronic device recognizes the direction of gravity and measures the plane position of the subject's face; The subject is instructed to set the position of the face plane as the reference plane before the exercise begins; The electronic device may instruct the subject to raise their head and measures the angle between the face plane and the reference plane; When the angle becomes greater than a certain value in the opposite direction to gravity, the electronic device determines that the subject has raised their head (FIG. 24).

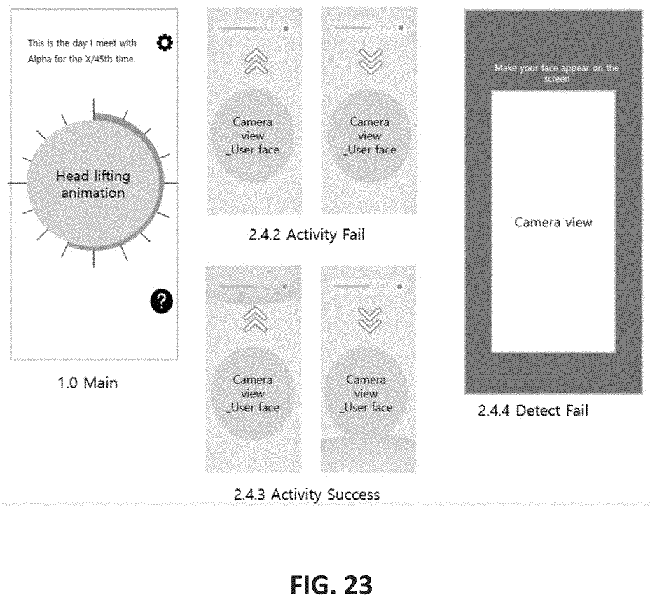

FIG. 23 is a diagram illustrating an exemplary usage flow of a digital application of the head lifting exercise of the present disclosure. At the start of the digital application, the subject may be instructed to perform head lifting exercises as an exemplary exercise of the voluntary skeletal muscle exercise modules. The digital application may instruct the subject to pick up the electronic device and position the electronic device on a cradle. The digital application may display a video of an animated head moving up and down and instruct the subject to follow the head motion of the animated head. The digital application may instruct the subject to click the activity start button and activate the timer that measures the progress of the exercise module and the remaining time of the exercise module. The digital application may display the subject's head on the electronic device screen and recognize the subject's head lifting motion; if the digital application does not recognize the subject's head lifting motion, the application may output a “please recognize your face” message. The digital application may end the activity when the timer reaches 0.

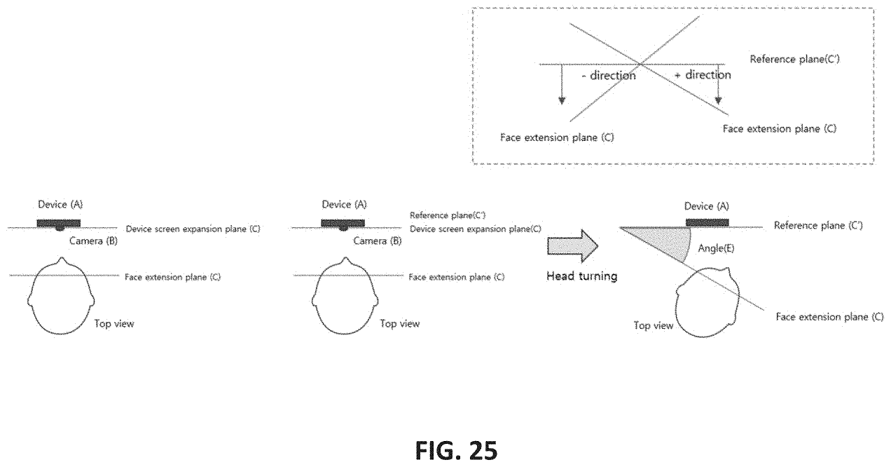

In certain embodiments, wherein the voluntary skeletal muscle exercise module comprises a head turning exercise instruction to turn a head of the subject clockwise and/or counterclockwise at a predetermined angle from the reference plane. FIG. 25 is a diagram illustrating the head turning sensing configuration for the voluntary skeletal muscle exercise module. In certain embodiments, the electronic device recognizes the subject's face through the camera of the electronic device; the electronic device sets the plane that extends the device screen, and measures the position of the plane of the subject's face; before the activity starts, the position of the extended plane of the device screen may be set as the reference plane; the electronic device may determine that the subject's face has rotated to the right when the subject's face plane moves more than a certain angle in the clockwise direction compared to the reference plane (FIG. 25). The electronic device may determine that the subject's face has rotated to the left when the subject's face plane moves more than a certain angle in the counterclockwise direction compared to the reference plane (FIG. 25).

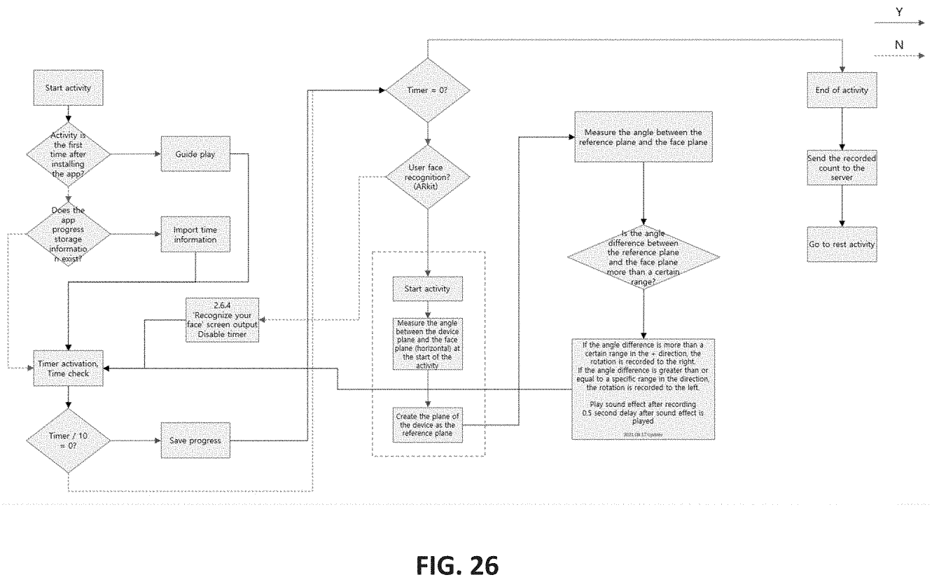

FIG. 26 is a flow chart illustrating an exemplary execution flow for the head turning instructions for the voluntary muscle exercise module. At the start of the digital application, the subject may be instructed to perform the head turning sensing configurations if this is the subject's first time installing the digital application. The digital application may load the application progress storage information if this is not the first time the subject has used the digital application. The digital application may import time and data information from previous stored data and activate a timer to save the subject's progress for the head turning exercise module. The digital application may also begin a user face recognition process before the start of the head turning activity by instructing the subject to perform the head turning sensing configurations. At the end of the head turning exercise module, the digital application may save the recorded data to the server and launch the rest module.

In certain embodiments, wherein the voluntary skeletal muscle exercise module comprises a hand gripping exercise instruction to grip the fist of the subject at a rate matching a displayed animation on the electronic device. In certain embodiments, the electronic device recognizes the subject's hand with the camera on the electronic device and saves the image every 0.01 seconds; the electronic device may measure the rate of the subject's open fist motion and closed fist motion compared to the displayed animations of an open fist and a closed fist on the electronic device; the electronic device may determine that the subject has performed the open fist and closed fist motion of the exercise when the photo stored for the open fist motion does not match the subject's current hand motion.

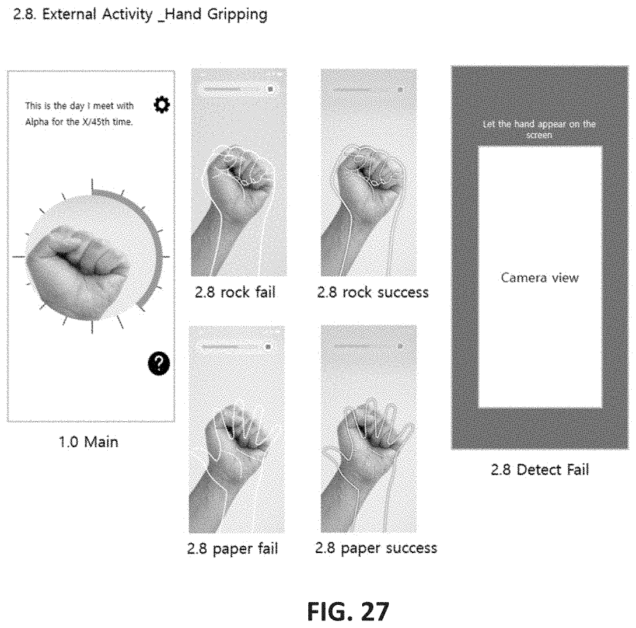

FIG. 27 is a diagram illustrating an exemplary usage flow of a digital application of the hand gripping exercise of the present disclosure. At the start of the digital application, the subject may be instructed to perform hand gripping exercises as an exemplary exercise of the voluntary skeletal muscle exercise modules. The digital application may instruct the subject to close their fists as the “rock” model animation and open their fists as the “paper” model animation while following the animated outline of a hand on the screen of the electronic device. The digital application may record the subject's hand gripping motions with the front facing camera of the electronic device. The digital application may instruct the subject to reposition their hands if the camera fails to detect the subject's hand. The digital application may end the activity when the timer reaches 0.

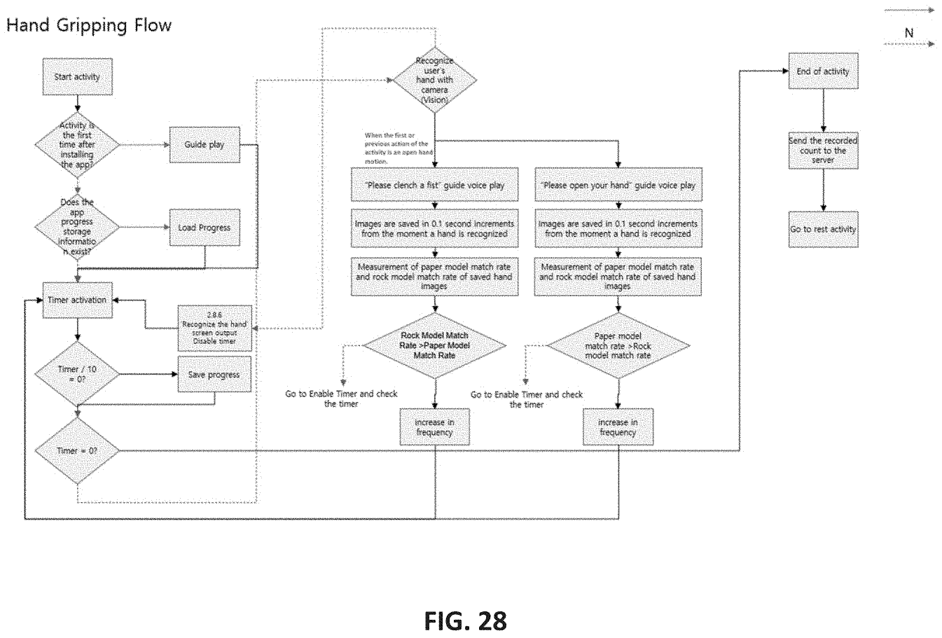

FIG. 28 is a flow chart illustrating an exemplary execution flow for the hand gripping instructions for the voluntary muscle exercise module. At the start of the digital application, the subject may be instructed to perform the hand gripping configurations if this is the subject's first time installing the digital application. The digital application may load the application progress storage information if this is not the first time the subject has used the digital application. The digital application may import time and data information from previous stored data and activate a timer to save the subject's progress for the hand gripping exercise module. The digital application may also begin a user hand recognition process before the start of the hand gripping exercise by instructing the subject to perform the hand gripping sensing configurations. The digital application may also recognize the subject's hand through the front facing camera of the electronic device and instruct the subject to “please clench a fist” or “please open your hand” using a guide voice. The digital application may save images in about 0.1 second increments from the moment the subject's hand is recognized. The digital application may measure the subject's rate of fist clenching with the saved images captured in about 0.1 second increments. The digital application may increase the frequency of the displayed animation instructing the subject to open and close their hand. At the end of the hand gripping exercise module, the digital application may save the recorded data to the server and launch the rest module.

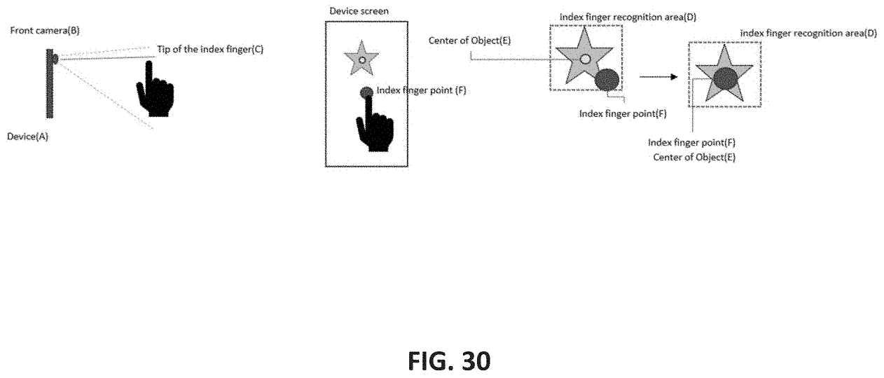

In certain embodiments, wherein the voluntary skeletal muscle exercise module comprises an arm shaking exercise instruction to move an object displayed on the electronic device across the screen with an index finger of the subject. FIG. 30 is a diagram illustrating an exemplary arm shaking exercise configuration for the voluntary skeletal muscle exercise module. In certain embodiments, the electronic device recognizes the subject's tip of the index finger with the front camera on the electronic device; On the screen of the electronic device, an object image in an arbitrary area, and a square-shaped index finger recognition area having a size that may include the object image are displayed; When the subject's index finger touches the index finger recognition area, the center of the displayed object image is moved to the position of the subject's index finger; As the subject moves their index finger, the object image follows on the screen of the electronic device; The electronic device measures the distance the object image has moved and sends the data to the doctor's web portal (FIG. 30).

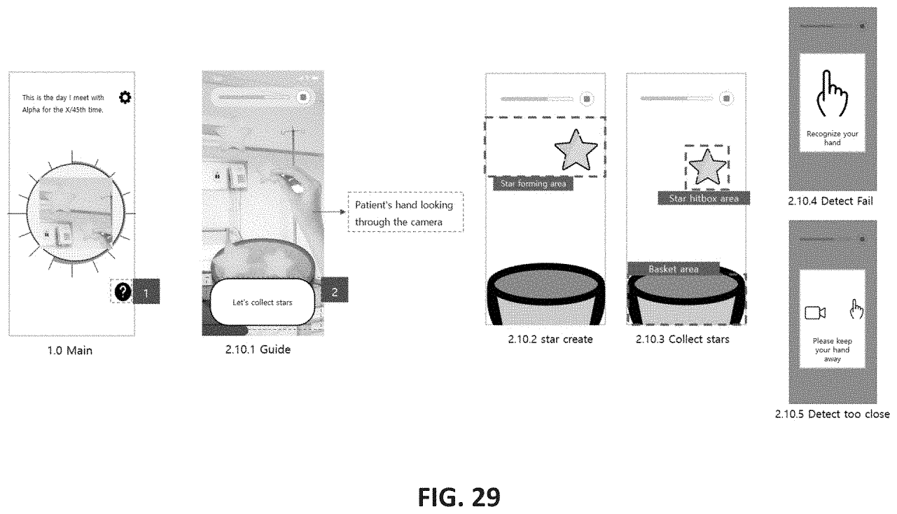

FIG. 29 is a diagram illustrating an exemplary usage flow of a digital application of the arm shaking exercise of the present disclosure. At the start of the digital application, the subject may be instructed to perform arm shaking exercises as an exemplary exercise of the voluntary skeletal muscle exercise modules. The digital application may instruct the subject to use their index fingers to move animated objects across the screen of the electronic device. The digital application may record the subject's arm shaking motions with the front facing camera of the electronic device. The digital application may instruct the subject to reposition their hands if the camera fails to detect the subject's hand. The digital application may end the activity when the timer reaches 0.

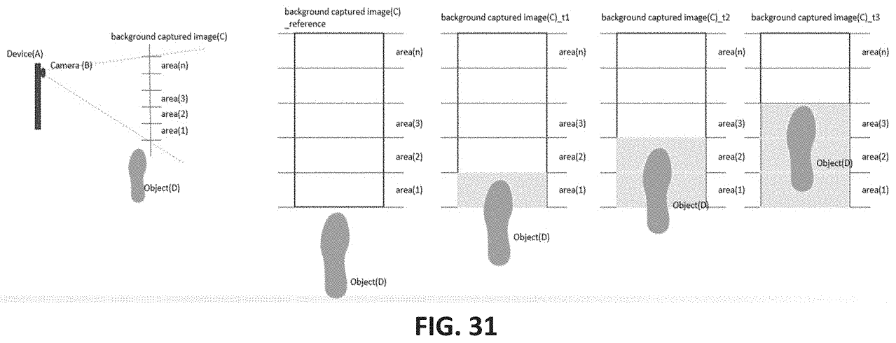

In certain embodiments, wherein the voluntary skeletal muscle exercise module comprises a leg lifting exercise instruct to lift their legs as measured by a change in a background area captured by the electronic device. FIG. 31 is a diagram illustrating an exemplary leg lifting exercise configuration for the voluntary skeletal muscle exercise module. In certain embodiments, the electronic device captures the background image of the subject and divide the image into multiple areas; the electronic device assigns an RGB (red, green, blue) value to each area and sets the background image as a reference value (FIG. 31). The electronic device determines that the subject has placed their foot in the corresponding area when the average RGB values of various areas of the image photographed at a specific time is different compared to the reference value of the background image (FIG. 31). The electronic device measures the movement of the subject when the amount of change in a specific area is sequentially greater than the reference value of the background image.

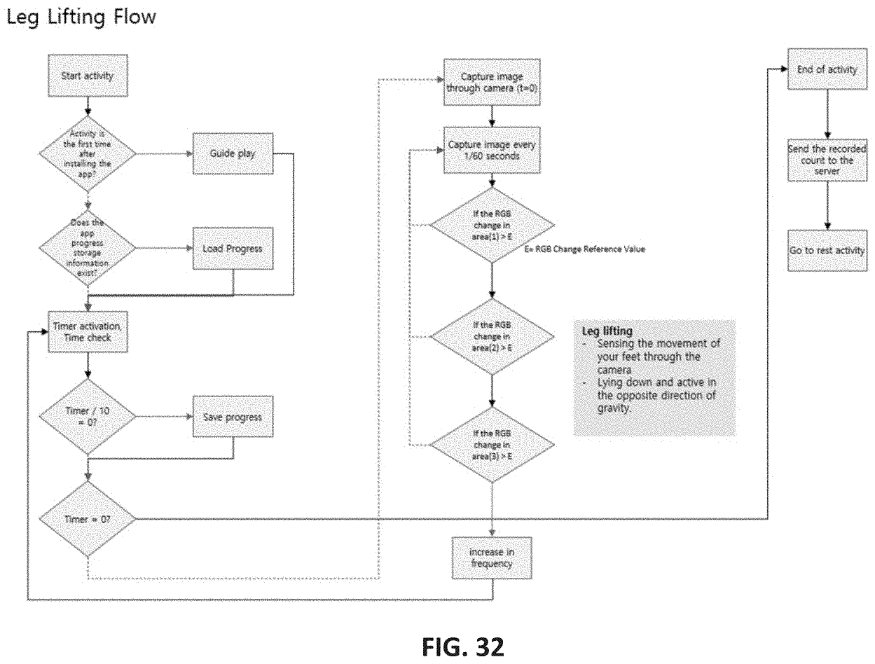

FIG. 32 is a flow chart illustrating an exemplary execution flow for the leg lifting exercise instructions for the voluntary muscle exercise module. At the start of the digital application, the subject may be instructed to perform the leg lifting exercise configurations if this is the subject's first time installing the digital application. The digital application may load the application progress storage information if this is not the first time the subject has used the digital application. The digital application may import time and data information from previous stored data and activate a timer to save the subject's progress for the leg lifting exercise module. The digital application may also begin a leg lifting recognition process before the start of the leg lifting exercise by instructing the subject to perform the leg lifting sensing configurations. The digital application may also recognize the subject's leg through the front facing camera of the electronic device and assign reference RGB values to the subject's background image. The digital application may save images in about 0.1 second increments from the moment the subject's leg is recognized. The digital application may measure the subject's rate of leg lifting with the saved images captured in about 0.1 second increments. The digital application may increase the frequency of the displayed animation instructing the subject to lift their leg if the change in RGB values is greater than reference RGB values. At the end of the leg lifting exercise module, the digital application may save the recorded data to the server and launch the rest module.

In certain embodiments, the one or more first instructions comprise instructions for aerobic, resistance, and/or concurrent exercise. Physical exercise (e.g., cardiovascular exercise) can promote IGF-1 production, which can (i) inhibit myostatin and prevent muscular atrophy associated with proteolysis, and/or (ii) activate insulin receptor substrate 1 (IRS1)-PI3K-AKT signaling and AKT to promote proteosynthesis. A module comprising physical exercise can include, for example, 20 minutes of high intensity exercise and 40 minutes of rest or low intensity exercise in a patient-bearable situation (for example, a module that mimics a low mountain climb, walk, etc.). This module can be repeated daily, and the effects observed after 1 week, 2 weeks, 3 weeks, 1 month, 2 months, 3 months, 4 months, 5 months, 6 months, 9 months, or 1 year.

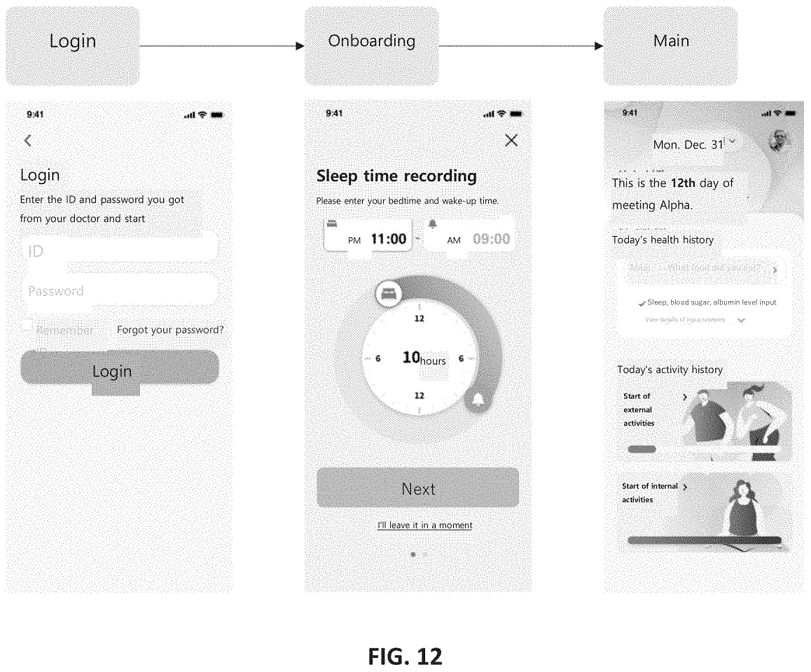

In certain embodiments, the one or more first modules comprise of the subject inputting health information into the electronic device. FIG. 12 is a diagram illustrating an exemplary patient portal structure of a digital application of the onboarding process of the present disclosure. In certain embodiments, the health information may comprise of the subject's sleep schedule, blood sugar levels, protein, amino acid, anti-inflammatory health food intake information, appetite stimulant consumption information, and albumin levels. In certain embodiments, the subject may input the health information as an onboarding process (FIG. 12). In certain embodiments, the digital application may instruct the subject to input their stable heart rate measurement. In certain embodiments, a subject's stable heart rate measurement may be a subject's resting heart rate measured at the initial onboarding process. In certain embodiments, the health information may be compiled into a daily checklist by the digital application wherein, the subject may be instructed to input their sleep and wake up times, blood sugar values, daily food intake, albumin levels, performance of daily external exercises, and performance of internal activities. In certain embodiments, the digital application may instruct the subject to input sleep and wake up times to measure whether the subject has enough sleep. In certain embodiments, a subject may be instructed to input blood sugar levels on an empty stomach before breakfast. In certain embodiments, the digital application may instruct the subject to input daily food intake to measure whether the subject is consuming enough protein, anti-inflammatory health food, and appetite stimulant. In certain embodiments, the digital application may instruct the subject to input albumin levels. In certain embodiments, the normal range of the subject's albumin levels may be about 3.5-5.2 g/dL. In certain embodiments, external activities may comprise of voluntary skeletal muscle exercises or aerobic exercises. In certain embodiments, internal activities may comprise of mediation exercises, breathing exercises, or massage exercises.

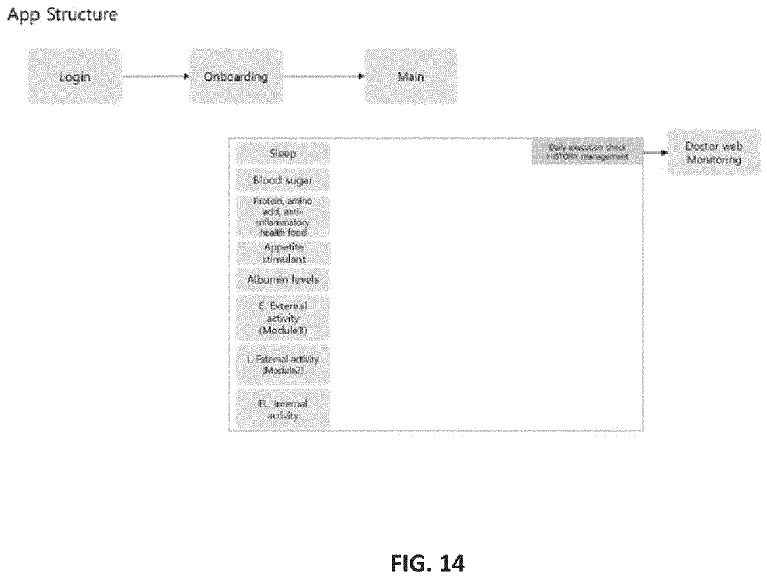

FIG. 14 is a flow chart illustrating an exemplary patient portal structure of the present disclosure. At the start of the application, the subject may be instructed to login with a “subject information key” automatically generated to subjects by healthcare providers. The health care provider may be instructed to input patient information before the subject's onboarding process. In addition, the subject may be instructed to onboard the application by inputting sleep and wake schedules, food intake information, and resting heart rate. In addition, the application records the subject's sleep and wake schedule, blood sugar, food intake information, albumin levels, and may instruct the subject to perform aerobic exercises, voluntary skeletal muscle exercises, and relaxation exercises.

In certain embodiments, the one or more first modules comprise the vagal nerve stimulation module, and the vagal nerve stimulation module comprises one or more first instructions to reduce inflammation in the subject. In certain embodiments, the one or more first modules comprise the vagal nerve stimulation module, and the vagal nerve stimulation module comprises at least one instruction selected from the group consisting of sense stimulation instructions for sight, sound, touch, taste, and smell. In certain embodiments, the vagal nerve stimulation module comprises one or more sense stimulation instructions for sight. In certain embodiments, the one or more sense stimulation instructions for sight include one or more instructions to view one or more figures or photos to stimulate autonomic nervous system. In certain embodiments, the vagal nerve stimulation module comprises one or more sense stimulation instructions for sound. In certain embodiments, the one or more sense stimulation instructions for sound include one or more instructions to hear one or more sounds to cause horror or relaxation. In certain embodiments, the vagal nerve stimulation module comprises one or more sense stimulation instructions for touch. In certain embodiments, the one or more sense stimulation instructions for touch include one or more instructions for abdominal breathing, controlling rate of breathing, cold massage, coughing, and skin massage. In some embodiments, the cold massage includes decreasing a temperature of a face of the subject. In certain embodiments, the vagal nerve stimulation module comprises one or more sense stimulation instructions for touch. In certain embodiments, the one or more sense stimulation instructions for touch include one or more instructions for abdominal breathing. In certain embodiments, the vagal nerve stimulation module comprises one or more sense stimulation instructions for taste. In certain embodiments, the one or more sense stimulation instructions for taste include one or more instructions to eat food to stimulate digestive glands in the subject. In certain embodiments, the vagal nerve stimulation module comprises one or more sense stimulation instructions for smell. In certain embodiments, the one or more sense stimulation instructions for smell include one or more instructions to stimulate digestive glands and/or to relax.

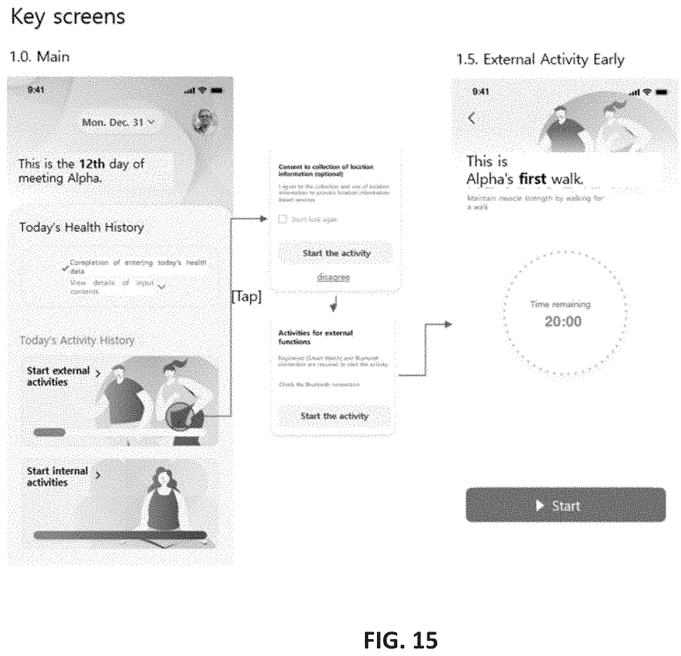

In certain embodiments, the one or more first modules comprise the aerobic exercise module, and the aerobic exercise module comprises one or more first instructions to increase adiponectin secretion in the subject. In certain embodiments, the one or more first instructions comprise one or more instructions for walking, biking, aerobic dance and/or swimming. FIG. 15 is a diagram illustrating an exemplary digital application structure that instructs the start of an external activity comprise, for example, aerobic exercises in about 30, 20, 10, or 5 minutes.

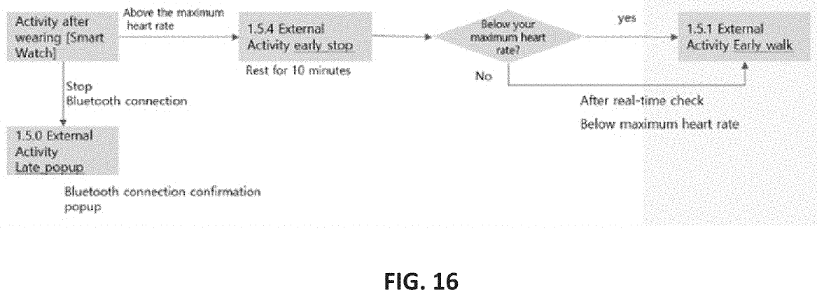

In certain embodiments, the one or more first modules may comprise of a guide screen that indicates the subject needs rest when maximum heart rate is reached. FIG. 16 is a flow chart illustrating the target heart rate calculation formula of a digital application of the present disclosure. In certain embodiments, the maximum heart rate may be calculated as [{207−(0.7*age)}-basal heart rate]*0.7+basal heart rate by the electronic device (FIG. 16). In certain embodiments, basal heart rate may be the subject's stable heart rate when setting up the device for the first time. The target heartrate of the subject may be kept within a medium intensity range of 50-70%. If the subject exceeds the maximum heart rate, the digital application may instruct the subject to stop the aerobic or voluntary skeletal muscle exercises (FIG. 16). In certain embodiments, the electronic device detects abnormal heart rate from the subject and a guide screen indicates that the subject should sufficiently rest. In certain embodiments, sufficient rest may be about 30, 20, 10 or 5 minutes, or whenever the subject's heart rate falls below the maximum heart rate. In certain embodiments, once the subject's heart rate falls below the maximum heart rate, the digital application may instruct the subject to resume the aerobic or voluntary muscle exercises (FIG. 16). In certain embodiments, after the subject's heart rate falls below the maximum heart rate, the subject may choose to end the aerobic exercise module or add one more aerobic exercise module. In certain embodiments, a guide screen instructing the subject to rest may be presented after the completion of the aerobic exercise module. In certain embodiments, the aerobic exercise module for late-stage subjects may be scheduled for about 30, 20, 10, or 5 minutes per day for a total of about 90 days.

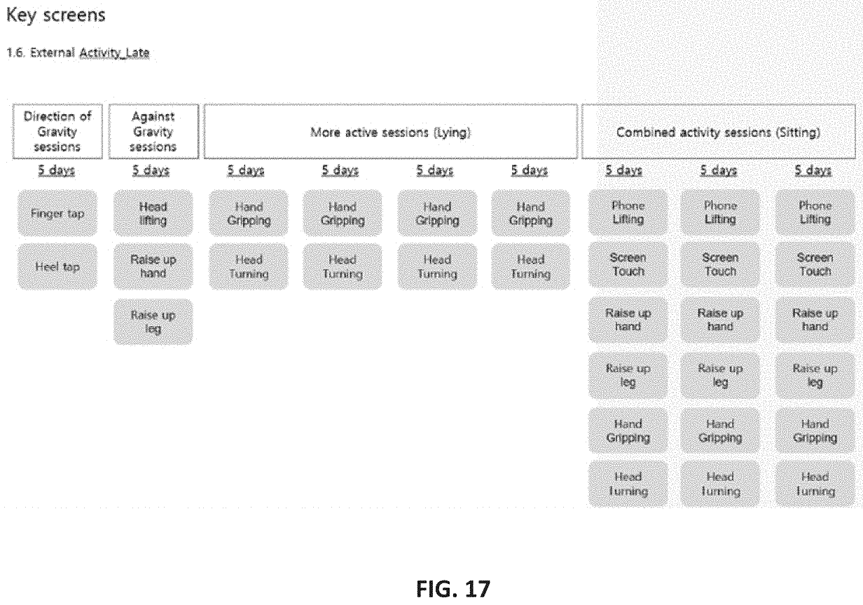

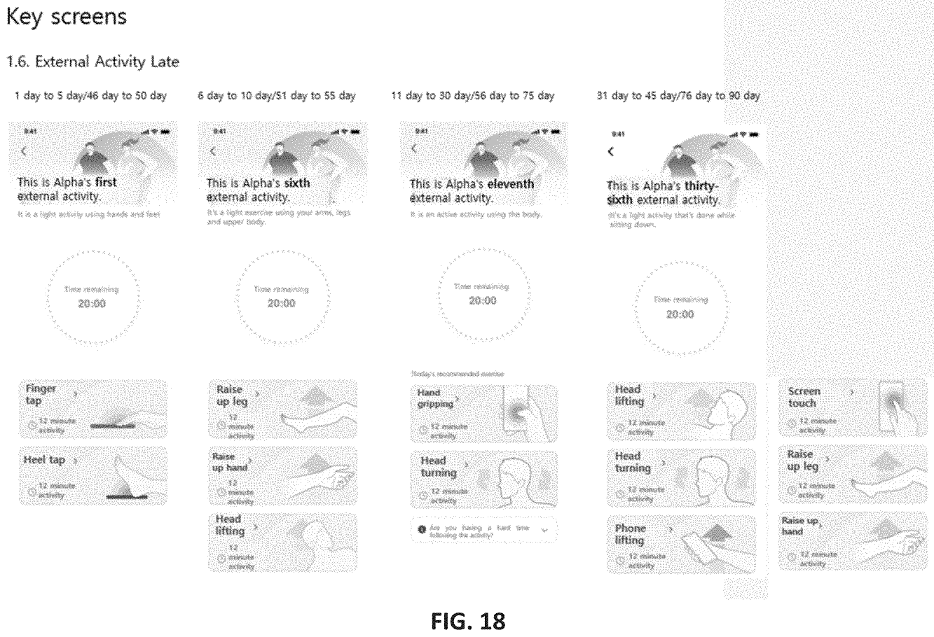

In certain embodiments, the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions (FIG. 17). In certain embodiments, the gravity exercise sessions may comprise one or more instructions for finger taps and heel taps (FIG. 18). In certain embodiments, the finger taps exercise may instruct the subject to lie down and touch the electronic device screen with their finger for about 5, 4, 3, 2, or 1 minute, then rest for 3, 2, or 1 minute by deep breathing. In addition, the electronic device may provide sound feedback when the subject inputs the screen touch. In certain embodiments, the heel taps exercise may instruct the subject to lie down and touch the electronic device screen with their heels for about 5, 4, 3, 2, or 1 minute, then rest for 3, 2, or 1 minute by deep breathing. In addition, the electronic device may provide sound feedback when the subject inputs the heel screen touch.

In certain embodiments, one or more first instructions for anti-gravity exercise sessions comprise one or more instructions for head lifting, raising up hand, and raising up leg (FIG. 18). In certain embodiments, the head lifting exercise may instruct the subject to lie down and raise their head according to the guide instructions for about 5, 4, 3, 2, or 1 minute. In certain embodiments, the guide instructions may instruct the subject to raise their head for about 15, 10, 5, or 3 seconds and rest with deep breathing exercises for about 20, 15, 10, 8, or 6 seconds. In certain embodiments, the guide instructions for head lifting exercises may comprise voluntary breaks initiated by the subject at about 30, 20, 15, 10 and 5 seconds. In certain embodiments, the raising up hand exercise may instruct the subject to lie down and raise their left or right hand according to the guide instructions for about 5, 4, 3, 2, or 1 minute. In certain embodiments, the guide instructions may instruct the subject to raise their left or right hand for about 15, 10, 5, or 3 seconds and rest with deep breathing exercises for about 20, 15, 10, 8, or 6 seconds. In certain embodiments, the guide instructions for hand lifting exercises may comprise voluntary breaks initiated by the subject at about 30, 20, 15, 10 and 5 seconds. In certain embodiments, the raising up leg exercise may instruct the subject to lie down and raise their left or right leg according to the guide instructions for about 5, 4, 3, 2, or 1 minute. In certain embodiments, the guide instructions may instruct the subject to raise their left or right leg for about 15, 10, 5, or 3 seconds and rest with deep breathing exercises for about 20, 15, 10, 8, or 6 seconds. In certain embodiments, the guide instructions for raising up leg exercises may comprise voluntary breaks initiated by the subject at about 30, 20, 15, 10 and 5 seconds.

In certain embodiments, one or more first instructions for lying down exercise sessions may comprise one or more instructions for hand gripping, fist clenching, and head turning (FIG. 18). In certain embodiments, the hand gripping exercise may instruct the subject to lie down and grab the phone and press the screen for about 5, 4, 3, 2, or 1 second with their thumb and then rest by deep breathing for about 15, 10, 5, or 3 second s. In certain embodiments, after the subject has performed the hand gripping exercise for about 5, 4, 3, 2, or 1 minute, the subject may be instructed to rest for about 3, 2, or 1 minute by deep breathing. In certain embodiments, the fist clenching exercise may instruct the subject to lie down and slowly open and close their fists for about 15, 10, 5, or 3 seconds and then rest by deep breathing for about 15, 10, 5, or 3 seconds. In certain embodiments, after the subject has performed the hand gripping exercise for about 5, 4, 3, 2, or 1 minute, the subject may be instructed to rest for about 3, 2, or 1 minute by deep breathing. In certain embodiments, the head turning exercise may instruct the subject to lie down and slowly turn their head for about 15, 10, 5, or 3 seconds and then rest by deep breathing for about 15, 10, 5, or 3 seconds. In certain embodiments, after the subject has performed the head turning exercise for about 5, 4, 3, 2, or 1 minute, the subject may be instructed to rest for about 3, 2, or 1 minute by deep breathing.

In certain embodiments, one or more first instructions for sitting exercise sessions may comprise one or more instructions for phone lifting exercise, screen touching exercises, raising up hands exercise, raising up legs exercise, hand gripping exercise, and head turning exercise while the subject is sitting down (FIG. 18).

In certain embodiments, the voluntary skeletal muscle exercise module may be scheduled for about 60, 45, 30, or 15 days (FIG. 17). In certain embodiments, the voluntary skeletal muscle exercise module may be repeated and scheduled for a total of about 120, 90, 60, or 30 days (FIG. 17). In certain embodiments, one or more first instructions for gravity exercise sessions may be scheduled for about 15, 10, 5, or 3 days (FIG. 18). In certain embodiments, one or more first instructions for anti-gravity exercise sessions are scheduled for about 15, 10, 5, or 3 days (FIG. 17). In certain embodiments, one or more first instructions for lying down exercise sessions may be scheduled for about 30, 25, 20 or 15 days (FIG. 17). In certain embodiments, one or more first instructions for sitting down exercise sessions may be scheduled for about 20, 15, 10 or 5 days (FIG. 17). In certain embodiments, one or more instructions for gravity exercise sessions, anti-gravity exercise sessions, lying down exercise sessions, and sitting exercise sessions may be scheduled for about 30, 25, 20, 15, or 10 minutes per session each day (FIG. 18). In certain embodiments, the electronic device may display a guide screen indicating that the subject needs rest when maximum heart rate is reached.

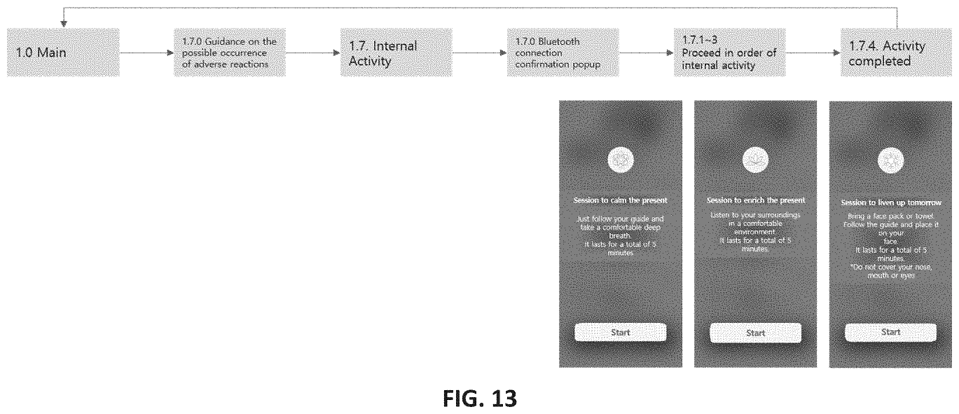

In certain embodiments, the one or more first modules comprise the relaxation module, and the relaxation module comprises one or more first instructions to increase adiponectin secretion in the subject. In certain embodiments, the one or more first instructions to increase adiponectin secretion in the subject comprise one or more meditation instructions. In certain embodiments, the one or more first instructions to increase adiponectin secretion in the subject comprise one or more sound instructions to hear relaxing sound. In some embodiments, the one or more sound instructions include generating relaxing sound, for example, including white noise. In additional embodiments, the device described herein comprises a speaker configured to generate the relaxing sound. One mechanism by which adiponectin can be increased is vagal nerve stimulation, which produces an anti-inflammatory effect and proteolysis block. In some embodiments, the relaxation module comprises meditation. In some embodiments, the relaxation module may instruct the subject to meditate by listening to white noise in a comfortable environment for about 10, 7, 5, or 3 minutes. In some embodiments, the relaxation module comprises deep breathing. In some embodiments, the relaxation module may instruct the subject to slowly breath in for about 10, 7, 5, or 3 seconds and exhale for 5, 3, or 1 second. In some embodiments, the relaxation module comprises massage exercises. In some embodiments, the relaxation module may instruct the subject to bring a face pack or towel and place it on the subject's face without covering the nose, mouth or eyes for about 10, 7, 5, or 3 minutes. In some embodiments, the relaxation module enables abdominal nerve stimulation. In some embodiments, the relaxation module is performed in an atmosphere or environment that is relaxing to the patient. In some embodiments, the relaxation module comprises listening to music. In some embodiments, the relaxation modules are scheduled for about 30, 20, 10 or 5 minutes. This module can be repeated daily, and the effects observed after 1 week, 2 weeks, 3 weeks, 1 month, 2 months, 3 months, 4 months, 5 months, 6 months, 9 months, or 1 year. FIG. 13 is a flow chart illustrating an exemplary internal activity use flow of a digital application of the meditation, breathing, and massage modules in the present disclosure. The subject may be instructed in the internal activity modules of the digital application to perform breathing exercises, mediation exercises, or massage exercises for a duration of about 30, 20, 10 or 5 minutes.

A session may comprise any number of digital therapeutic modules. In some embodiments, a session may comprise two or more digital therapeutic modules. In some embodiments, a session may comprise 3 or more, 4 or more, 5 or more, 6 or more, 7 or more, 8 or more, 9 or more, 10 or more, 11 or more, 12 or more, 13 or more, 14 or more, 15 or more, 20 or more, or 25 or more digital therapeutic modules. A session may comprise any number of digital therapeutic modules, and the digital therapeutic modules may be independently selected from a voluntary skeletal muscle exercise module, a vagal nerve stimulation module, an aerobic exercise module, and a relaxation module. In some embodiments, a session may consist of 4 digital therapeutic modules, and the digital therapeutic modules comprise a voluntary skeletal muscle exercise module, a vagal nerve stimulation module, an aerobic exercise module, and a relaxation module. A person of skill in the art will appreciate that there are a vast number of combinations for the number and type(s) of digital therapeutic modules that may go into a particular session. A session can be repeated as frequently or as infrequently as needed. In some embodiments, a session can be repeated 5 times per day, 4 times per day, 3 times per day, 2 times per day, daily, every 2 days, every 3 days, every 4 days, every 5 days, every 6 days, every 7 days, every 2 weeks, every 3 weeks, or every 4 weeks.

For patients having late stage cancer, it is contemplated that a voluntary skeletal muscle exercise module can cause the patient harm. Accordingly, in certain embodiments of the present disclosure, if a subject has late stage cancer, a voluntary skeletal muscle exercise module is excluded from the method or system for treating cancer cachexia. In certain embodiments of the present disclosure, if a subject has a cancerous mass that is that is greater than or equal to 0.1 centimeter (cm), greater than or equal 0.5 cm, greater than or equal 1 cm, greater than or equal 2 cm, greater than or equal 3 cm, greater than or equal 4 cm, or greater than or equal 5 cm in diameter, a voluntary skeletal muscle exercise module is excluded from the method or system for treating cancer cachexia.

In some embodiments, the subject has moderate muscle atrophy, and the one or more first modules consists of the voluntary skeletal muscle exercise module, the vagal nerve stimulation module, the aerobic exercise module, and the relaxation module. In some embodiments, the subject has moderate muscle atrophy, and the one or more first modules consists of the vagal nerve stimulation module, the aerobic exercise module, and the relaxation module.

In some embodiments, the subject has severe muscle atrophy and the method excludes providing a voluntary skeletal muscle exercise module and further excludes providing an aerobic exercise module.

In some embodiments, the external reviewer comprises a health professional (e.g., a healthcare provider or doctor). In some embodiments, the external reviewer comprises an artificial intelligence (AI). The term “artificial intelligence” can refer to intelligence exhibited by machines. In computer science, an ideal “intelligent” machine is a flexible rational agent that perceives its environment and takes actions that maximize its chance of success at some goal (e.g., treating cancer cachexia in a patient). Colloquially, the term “artificial intelligence” is applied when a machine mimics “cognitive” functions that humans associate with other human minds, such as “learning” and “problem solving. The term artificial intelligence may refer to an algorithm that may apply learning skills on multiple types of information (such as physiological information, additional information and person's medical history).

In some embodiments, the digital apparatus comprises a sensor, and the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, a thermometer, a gesture recognition, and a face recognition. In some embodiments, the sensor comprises a touch sensor, and the subject provides the adherence information to the electronic device using the touch sensor.

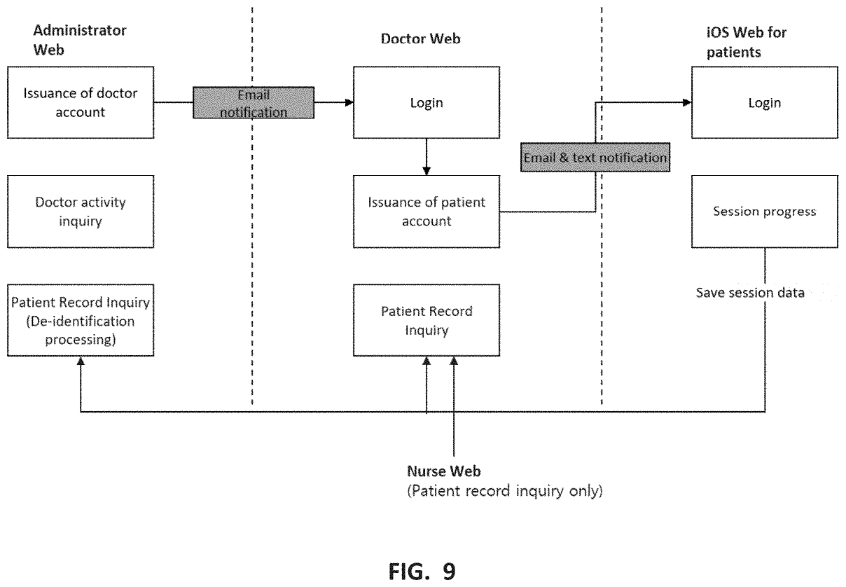

FIG. 9 depicts a flow chart illustrating a system for treating cancer cachexia, the system comprising an administrative portal (e.g., Administrator's web), a healthcare provider portal (e.g., Doctor's web) and a digital apparatus configured to execute a digital application (e.g., an application or ‘app’) for treating cancer cachexia in a subject. Among other things, the Administrator's portal allows an administrator to issue doctor accounts, review doctor information, and review de-identified patient information. Among other things, the Healthcare Provider's portal allows a healthcare provider (e.g., a doctor) to issue patient accounts, and review patient information (e.g., age, prescription information, and status for having completed one or more digital therapeutic modules or sessions). Among other things, the digital application allows a patent access to complete one or more digital therapeutic modules or sessions.

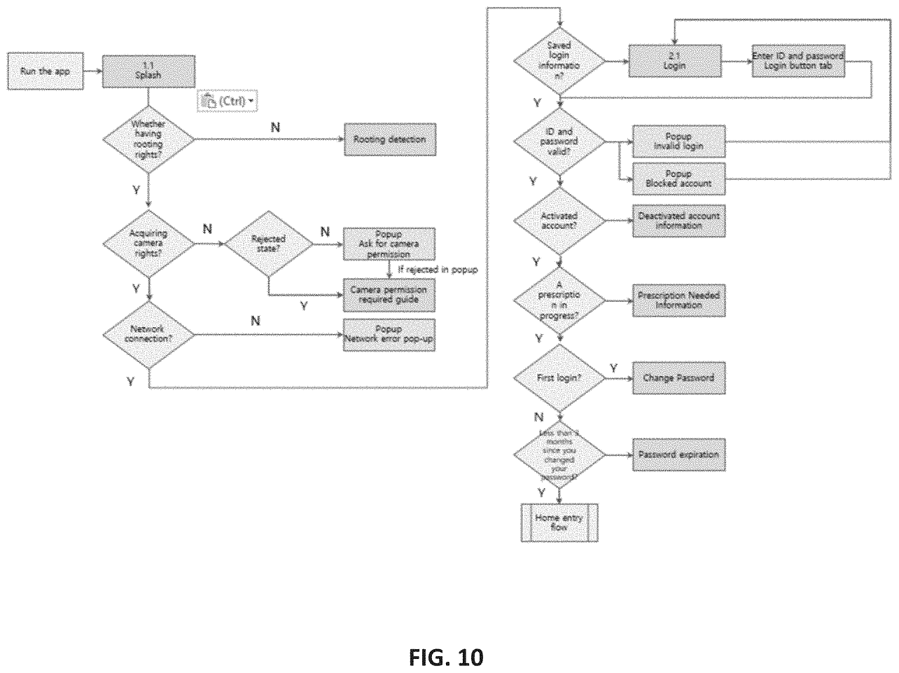

FIG. 10 is a flow chart illustrating an exemplary digital application usage flow of the present disclosure At the start of the application, a splash process may initiate the privacy and data access consent requests. The privacy and data access consent requests of the application may ask the subject to allow rooting rights, camera rights, and network connection rights on the electrical device. After consent is given to the application, the subject may be instructed to proceed with login verification. In addition, the login verification process may comprise, for example, the registration of ID and password, the activation of account, the confirmation of a prescription in progress, the option to change password if this is a first login, or the option to change password as a result of password expiration.

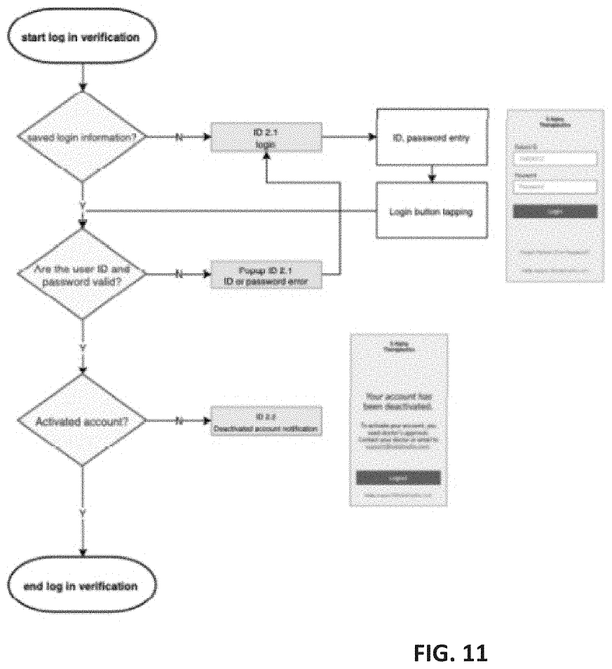

FIG. 11 depicts a flow chart illustrating an execution flow for login verification during a splash process at the starting of the digital application. In addition, a prescription verification process may comprise, for example, determining if the treatment period has expired, determining whether the subject has been recently (e.g., within the last hour) performed a voluntary skeletal muscle exercise), determining if, based on the prescription, the subject's sessions for the day have been completed (e.g., the subject is compliant with the prescription). In such instances, the digital apparatus may notify the subject that there are no sessions available to be completed.

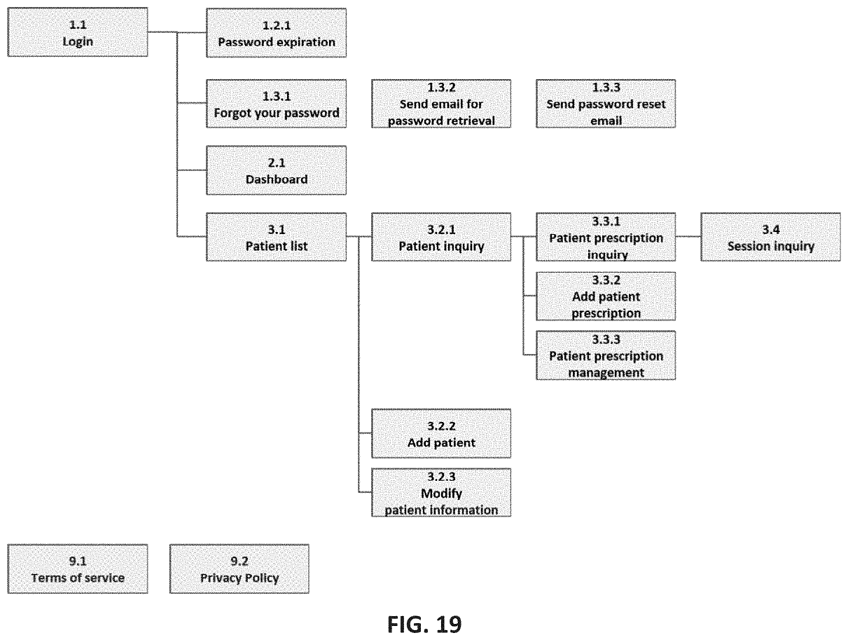

FIG. 19 is a flow chart illustrating an exemplary doctor portal structure and administrative portal structure of a digital application of the present disclosure. At the start of the digital application, the exemplary doctor portal may instruct the healthcare provider to login with a registered username and password. The digital application may instruct the healthcare provider to provide a new password if the healthcare provider's password has expired or the healthcare provider has forgotten the password. The digital application may provide a password retrieval process by sending an email to the healthcare provider. After the healthcare provider logs into the digital application, the dashboard of the digital application may provide a patient list, patient inquiries, patient prescription inquiries, and session inquiries. The digital application may allow the healthcare provider to add or modify patient information. The digital application may allow the healthcare provider to add patient prescription or manage patient prescriptions.

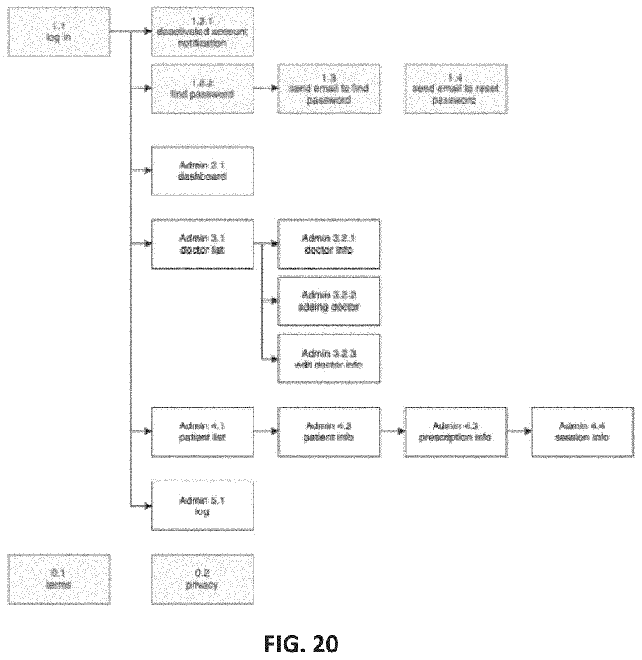

FIG. 20 is a flow chart illustrating an exemplary execution flow for an administrative portal in a system of the present disclosure. At the start of the digital application the exemplary administrative portal may instruct the administer to login with a registered username or password. The digital application may instruct the administrator to provide a password. If the administrator has forgotten the password, the digital application may provide a password retrieval process by sending an email to the administrator. After the administrator logs into the digital application, the dashboard of the digital application may provide a doctor list, patient list, and log of inputted data. The digital application may allow the administrator to review doctor information, add doctors to list of healthcare provider, and edit doctor information. The digital application may allow the administrator to review patient list, patient information, patient prescription information, and patient session information.

In some embodiments, the healthcare provider portal provides a healthcare provider with one or more options, and the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject. In some embodiments, the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject. In some embodiments, the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day. In some embodiments, the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, and an indicator of whether the scheduled or prescribed digital therapeutic module was fully or partially completed.

A dashboard of a healthcare provider portal will be described in more detail. The dashboard of a healthcare provider portal may include the number of all patients associated with the present doctor's account. A graph may be used to show the number of patients who have opened the digital application for patient per day in the most recent 90 days. The number of patients in progress may also be viewed. A graph may be used to show the number of patients who have completed the daily sessions per day in the most recent 90 days. The healthcare provider portal may include a patient tab displaying a list of patients by displaying, for example, Patient ID (the unique identification number temporarily given to each patient when adding them on the list), Patient Name, Search bar for searching by ID, Name, Email, Memo, etc., and Add New Patient button for adding new patients. The patient tab may further display detailed information on a given patient, for example, detailed patient information, a button for editing patient information, prescription information, a button for adding a new prescription, a progress status for different each prescription, and a button or link for sending an email to the patient. The patient tab in the healthcare provider portal may further have an option for adding a new patient, for example, a button for adding a new patient, and displaying an error message when required patient information has not been provided. The patient tab in the healthcare provider portal may further include options for editing information of an existing patient by providing, e.g., a button or link for resetting a password, a button for deleting a given patient, and a button for saving changes. In addition, detailed prescription information can be displayed for a given patient. For example, the patient tab in the healthcare provider portal may include or display a button for editing prescription information, the duration of the sessions attended by the patient or subject, and an overview the treatment progress. Seven days are represented as a line or row of 7 squares. For 12 weeks, each 6 weeks may be presented separately. Different colors may be used to discern session statuses (e.g., grey for sessions not started, red for sessions not attended, yellow for sessions partially attended, and green for sessions fully attended). In some embodiments, the administrative portal provides an administrator with one or more options, and the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider. In some embodiments, the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider. In some embodiments, the one or more options comprise the viewing or editing the de-identified information of the subject, and the de-identified information of the subject comprises one or more selected from the group consisting of an identification number for the subject, and the healthcare provider for the subject. In some embodiments, the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules. In some embodiments, the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, and an indicator of whether the scheduled or prescribed digital therapeutic module was fully or partially completed.

A dashboard of an administrative portal may show the number of doctors. A graph may be used to show the number of doctors that have visited the digital application per day in the most recent 90 days. The number of all patients associated with the any doctor's account may be displayed on the administrative portal. A graph may be used to show the number of patients who have opened the digital application for patient per day in the most recent 90 days. The number of patients in progress may also be viewed. A graph may be used to show the number of patients who have completed the daily sessions per day in the most recent 90 days. A doctor tab in an administrative portal may display a list of doctors, by displaying, e.g., a search bar for searching for various doctors by name, email, etc., a button for adding a new doctor, the doctor's ID, a button for viewing detailed doctor information, and s deactivated doctor accounts. The doctor tab in the administrative portal further display a list of patients being cared for by a given doctor, with patient-identifying information redacted (*). For instance, the doctor tab displays is the doctor's account information, a button for editing the doctor's account information, a list of patients being cared for by the doctor, a list of patient ID numbers, a link or button for sending the doctor a registration email, a notification that the doctor's account has been deactivated, which only appears for deactivated accounts, and redacted or de-identified patient information. The doctor tab in the administrative portal may have options for adding a new doctor, editing information of an existing doctor, including activating or deactivating a doctor's account, etc. Additionally, a patient tab in the administrative portal displays information for one or more patients, wherein sensitive information is redacted. For example, the patient tab in the administrative portal displays detailed patient or prescription information for a given patient or detailed prescription information for a given patient.

While the disclosure has been shown and described with reference to certain exemplary embodiments thereof, it will be understood by those skilled in the art that various changes in form and details may be made therein without departing from the spirit and scope of the disclosure as defined by the appended claims.

### CERTAIN EMBODIMENTS

Embodiment 1. A method of treating cancer cachexia in a subject in need thereof, the method comprising: providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; wherein the electronic device (i) comprises a sensor sensing adherence by the subject to the first instructions of the one or more first modules, (ii) transmits adherence information, based on the adherence, to a server, and (iii) receives one or more second instructions from the server based on the adherence information; and providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions.
Embodiment 2. The method according to Embodiment 1, wherein the one or more first modules comprise the voluntary skeletal muscle exercise module, and the voluntary skeletal muscle exercise module comprises one or more first instructions to increase MGF/IGF-1 secretion in the subject.
Embodiment 3. The method according to Embodiment 2, wherein the voluntary skeletal muscle exercise module comprises a screen touching exercise instruction to touch a randomly generated displayed animation on the electronic device.
Embodiment 4. The method according to Embodiment 2, wherein the voluntary skeletal muscle exercise module comprises a head lifting exercise instruction to lift and position a head of the subject at a predetermined angle from the reference plane.
Embodiment 5. The method according to Embodiment 2, wherein the voluntary skeletal muscle exercise module comprises a head turning exercise instruction to turn a head of the subject clockwise and/or counterclockwise at a predetermined angle from the reference plane.
Embodiment 6. The method according to Embodiment 2, wherein the voluntary skeletal muscle exercise module comprises a hand gripping exercise instruction to grip a fist of the subject at a rate matching a displayed animation on the electronic device.
Embodiment 7. The method according to Embodiment 2, wherein the voluntary skeletal muscle exercise module comprises an arm shaking exercise instruction to move an object displayed on the electronic device across the screen with an index finger of the subject.
Embodiment 8. The method according to Embodiment 2, wherein the voluntary skeletal muscle exercise module comprises a leg lifting exercise instruct to lift their legs as measured by a change in a background area captured by the electronic device.
Embodiment 9. The method according to Embodiment 2, wherein said one or more first instructions comprise instructions for aerobic, resistance, and/or concurrent exercise.
Embodiment 10. The method according to Embodiments 1-9, wherein the subject inputs health information into the electronic device.
Embodiment 11. The method according to Embodiment 10, wherein the health information comprises of the subject's sleep schedule, blood sugar levels, protein, amino acid, anti-inflammatory health food intake information, appetite stimulant consumption information, and albumin levels Embodiment 12. The method according to any one of Embodiments 1-11, wherein the one or more first modules comprise the vagal nerve stimulation module, and the vagal nerve stimulation module comprises one or more first instructions to reduce inflammation in the subject.
Embodiment 13. The method according to any one of Embodiments 1-12, wherein the one or more first modules comprise the vagal nerve stimulation module, and the vagal nerve stimulation module comprises at least one instruction selected from the group consisting of sense stimulation instructions for sight, sound, touch, taste, and smell.
Embodiment 14. The method according to Embodiment 13, wherein the vagal nerve stimulation module comprises one or more sense stimulation instructions for sight, and the one or more sense stimulation instructions for sight include one or more instructions to view one or more figures to stimulate autonomic nervous system.
Embodiment 15. The method according to Embodiment 14, wherein the electronic device receives and displays the figures.
Embodiment 16. The method according to any one of Embodiments 13-15, wherein the vagal nerve stimulation module comprises one or more sense stimulation instructions for sound, and the one or more sense stimulation instructions for sound include one or more instructions to hear one or more sounds to cause horror or relaxation.
Embodiment 17. The method according to Embodiment 16, wherein the electronic device receives and plays the sounds.
Embodiment 18. The method according to any one of Embodiments 13-17, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.
Embodiment 19. The method according to any one of Embodiments 13-18, wherein the vagal nerve stimulation module comprises one or more sense stimulation instructions for touch, and the one or more sense stimulation instructions for touch include one or more instructions for abdominal breathing.
Embodiment 20. The method according to any one of Embodiments 13-19, wherein the vagal nerve stimulation module comprises one or more sense stimulation instructions for taste, and the one or more sense stimulation instructions for taste include one or more instructions to eat food to stimulate digestive glands in the subject.
Embodiment 21. The method according to Embodiment 20, wherein the electronic device receives and display information related to the food.
Embodiment 22. The method according to any one of Embodiments 13-21, wherein the vagal nerve stimulation module comprises one or more sense stimulation instructions for smell, and the one or more sense stimulation instructions for smell include one or more instructions to stimulate digestive glands and/or to relax.
Embodiment 23. The method according to Embodiment 22, wherein the electronic device is configured to release a scent for aroma therapy.
Embodiment 24. The method according to any one of Embodiments 1-23, wherein the one or more first modules comprise the aerobic exercise module, and the aerobic exercise module comprises one or more first instructions to increase adiponectin secretion in the subject.
Embodiment 25. The method according to Embodiment 24, wherein said one or more first instructions comprise one or more instructions for walking, biking, aerobic dance and/or swimming.
Embodiment 26. The method according to Embodiment 25, wherein the wherein the electronic device displays a guide screen indicating that the subject needs rest when maximum heart rate is reached.
Embodiment 27. The method according to Embodiment 26, wherein the maximum heart rate is calculated as [{207−(0.7*age)}-basal heart rate]*0.7+basal heart Embodiment 28. The method according to Embodiment 27, wherein the basal heart rate is the subject's stable heart rate when setting up the device for the first time.
Embodiment 29. The method according to Embodiment 28, wherein when the device detects abnormal heart rate, a guide screen indicates that the subject should sufficiently rest.
Embodiment 30. The method according to Embodiment 29, wherein sufficient rest is about 10 minutes, or whenever the subject's heart rate falls below the maximum heart rate.
Embodiment 31. The method according to Embodiment 2, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.
Embodiment 32. The method according to Embodiment 31, wherein said one or more first instructions for gravity exercise sessions comprise one or more instructions for finger taps and heel taps.
Embodiment 33. The method according to Embodiment 31, wherein said one or more first instructions for anti-gravity exercise sessions comprise one or more instructions for head lifting, raising up hand, and raising up leg.
Embodiment 34. The method according to Embodiment 31, wherein said one or more first instructions for lying down exercise sessions comprise one or more instructions for hand gripping, fist clenching, and head turning.
Embodiment 35. The method according to Embodiment 31, wherein said one or more first instructions for sitting exercise sessions comprise one or more instructions for phone lifting, screen touching, raising up hands, raising up legs, hand gripping, and head turning.
Embodiment 36. The method according to Embodiment 2 or Embodiment 31, wherein voluntary skeletal muscle exercise module is scheduled for 45 days.
Embodiment 37. The method according to Embodiment 36, wherein the voluntary skeletal muscle exercise module is repeated and scheduled for a total of 90 days.
Embodiment 38. The method according to Embodiment 31 or 32, wherein one or more first instructions for gravity exercise sessions are scheduled for about 5 days.
Embodiment 39. The method according to Embodiment 31 or 33, wherein one or more first instructions for anti-gravity exercise sessions are scheduled for about 5 days Embodiment 40. The method according to Embodiment 31 or 34, wherein one or more first instructions for lying down exercise sessions are scheduled for about 20 days.
Embodiment 41. The method according to Embodiment 31 or 35, wherein one or more first instructions for sitting down exercise sessions are scheduled for about 15 days.
Embodiment 42. The method according to Embodiment 31, wherein the one or more instructions for gravity exercise sessions, anti-gravity exercise sessions, lying down exercise sessions, and sitting exercise sessions are scheduled for about 20 minutes per session each day.
Embodiment 43. The method according to Embodiment 42, wherein the electronic device displays a guide screen indicating that the subject needs rest when maximum heart rate is reached.
Embodiment 44. The method according to Embodiment 43, wherein the maximum heart rate is calculated as [{207−(0.7*age)}-basal heart rate]*0.7+basal heart rate.
Embodiment 45. The method according to Embodiment 44, wherein the basal heart rate is the subject's stable heart rate when setting up the device for the first time.
Embodiment 46. The method according to Embodiment 45, wherein when the device detects abnormal heart rate, a guide screen indicates that the subject should sufficiently rest.
Embodiment 47. The method according to Embodiment 46, wherein sufficient rest is about 10 minutes, or whenever the subject's heart rate falls below the maximum heart rate.
Embodiment 48. The method according to any one of Embodiments 1-47, wherein the one or more first modules comprise the relaxation module, and the relaxation module comprises one or more first instructions to increase adiponectin secretion in the subject.
Embodiment 49. The method according to Embodiment 48, wherein the one or more first instructions comprise one or more breathing instructions.
Embodiment 50. The method according to Embodiment 48 or 49, wherein said one or more first instructions comprise one or more sound instructions to hear relaxing sound.
Embodiment 51. The method according to Embodiment 50, wherein the device receives and plays the relaxing sound.
Embodiment 52. The method according to Embodiment 48, wherein said one or more first instructions comprise one or more meditation instructions.
Embodiment 53. The method according to Embodiment 48, wherein said one or more first instructions comprise one or more massage instructions.
Embodiment 54. The method according to any one of Embodiments 48-53, wherein said instructions are scheduled for about 5 minutes.
Embodiment 55. The method according to any one of Embodiments 1-54, wherein the subject is an early cancer patient.
Embodiment 56. The method according to any one of Embodiments 1-54, wherein the subject has a cancer mass having a diameter of 3 cm or less.
Embodiment 57. The method according to any one of Embodiments 1 and 12-54, wherein the subject is a late cancer patient, and the method excludes providing a voluntary skeletal muscle exercise module.
Embodiment 58. The method according to any one of Embodiments 1 and 12-54, wherein the subject has a cancer mass having a diameter of more than 3 cm, and the method excludes providing a voluntary skeletal muscle exercise module.
Embodiment 59. The method according to Embodiment 55 or 56, wherein the subject has moderate muscle atrophy, and the one or more first modules consists of the voluntary skeletal muscle exercise module, the vagal nerve stimulation module, the aerobic exercise module, and the relaxation module.
Embodiment 60. The method according to Embodiment 58 or 59, wherein the subject has moderate muscle atrophy, and the one or more first modules consists of the vagal nerve stimulation module, the aerobic exercise module, and the relaxation module.
Embodiment 61. The method according to any one of Embodiments 1-60, wherein the subject has moderate muscle atrophy (in spec, define the moderate muscle atrophy as still being able to walk).
Embodiment 62. The method according to any one of Embodiments 1, 12-48, and 48-58, wherein the subject has severe muscle atrophy, and the method excludes providing a voluntary skeletal muscle exercise module and further excludes providing an aerobic exercise module.
Embodiment 63. The method according to Embodiment 62, wherein the one or more first modules consists of the vagal nerve stimulation module and the relaxation module.
Embodiment 64. The method according to any according to any one of Embodiments 1-63, wherein the server receives the one or more second instructions from an external reviewer.
Embodiment 65. The method according to any one of Embodiments 64, wherein the external reviewer comprises a health professional.
Embodiment 66. The method according to any one of Embodiments 64, wherein the external reviewer comprises an artificial intelligence (AI).
Embodiment 67. The method according to any one of Embodiments 1-66, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.
Embodiment 68. The method according to any one of Embodiments 1-67, wherein the sensor comprises a touch sensor, and the subject provides the adherence information to the electronic device using the touch sensor.
Embodiment 69. A system for treating cancer cachexia in a subject, comprising: a digital apparatus configured to execute a digital application comprising one or more first modules, for treating cancer cachexia in a subject, wherein the digital apparatus comprises a sensor for sensing adherence by the subject to a first set of instructions of the one or more first modules; a healthcare provider portal configured to provide one or more options to a healthcare provider to perform one or more tasks to prescribe treatment for the cancer cachexia in the subject based on information received from the digital application; and an administrative portal configured to provide one or more options to an administrator of the system to perform one or more tasks to manage access to the system by the healthcare provider.
Embodiment 70. The system of Embodiment 69, wherein the digital application for treating cancer cachexia instructs a processor of the digital apparatus to execute operations comprising: generating digital therapeutic modules for treating cancer cachexia based on a mechanism of action in and a therapeutic hypothesis for the cancer cachexia.
Embodiment 71. The system of Embodiment 70, wherein the generating of the digital therapeutic modules comprises generating the digital therapeutic modules based on biochemical factors related to the cancer cachexia.
Embodiment 72. The system according to Embodiment 69, wherein the one or more first modules comprise the voluntary skeletal muscle exercise module, and the voluntary skeletal muscle exercise module comprises one or more first instructions to increase MGF/IGF-1 secretion in the subject.
Embodiment 73. The system according to Embodiment 72, wherein the voluntary skeletal muscle exercise module comprises a screen touching exercise instruction to touch a randomly generated displayed animation on the electronic device.
Embodiment 74. The system according to Embodiment 72, wherein the voluntary skeletal muscle exercise module comprises a head lifting exercise instruction to lift and position a head of the subject at a predetermined angle from the reference plane.
Embodiment 75. The system according to Embodiment 72, wherein the voluntary skeletal muscle exercise module comprises a head turning exercise instruction to turn a head of the subject clockwise and/or counterclockwise at a predetermined angle from the reference plane.
Embodiment 76. The system according to Embodiment 72, wherein the voluntary skeletal muscle exercise module comprises a hand gripping exercise instruction to grip a fist of the subject at a rate matching a displayed animation on the electronic device.
Embodiment 77. The system according to Embodiment 72, wherein the voluntary skeletal muscle exercise module comprises an arm shaking exercise instruction to move an object displayed on the electronic device across the screen with an index finger of the subject.
Embodiment 78. The system according to Embodiment 72, wherein the voluntary skeletal muscle exercise module comprises a leg lifting exercise instruct to lift their legs as measured by a change in a background area captured by the electronic device Embodiment 79. The system according to Embodiment 72, wherein said one or more first instructions comprise instructions for aerobic, resistance, and/or concurrent exercise.
Embodiment 80. The system according to any one of Embodiments 69-79, wherein the subject inputs health information into the electronic device.
Embodiment 81. The system according to Embodiment 80, wherein the health information comprises of the subject's sleep schedule, blood sugar levels, protein, amino acid, anti-inflammatory health food intake information, appetite stimulant consumption information, and albumin levels.
Embodiment 82. The system according to any one of Embodiments 69-81, wherein the one or more first modules comprise the vagal nerve stimulation module, and the vagal nerve stimulation module comprises one or more first instructions to reduce inflammation in the subject.
Embodiment 83. The system according to any one of Embodiments 69-82, wherein the one or more first modules comprise the vagal nerve stimulation module, and the vagal nerve stimulation module comprises at least one instruction selected from the group consisting of sense stimulation instructions for sight, sound, touch, taste, and smell.
Embodiment 84. The system according to Embodiment 83, wherein the vagal nerve stimulation module comprises one or more sense stimulation instructions for sight, and the one or more sense stimulation instructions for sight include one or more instructions to view one or more figures (define in spec to include specific photos) to stimulate autonomic nervous system.
Embodiment 85. The system according to Embodiment 84, wherein the electronic device receives and displays the figures.
Embodiment 86. The system according to any one of Embodiments 83-85, wherein the vagal nerve stimulation module comprises one or more sense stimulation instructions for sound, and the one or more sense stimulation instructions for sound include one or more instructions to hear one or more sounds to cause horror or relaxation.
Embodiment 87. The system according to Embodiment 86, wherein the electronic device receives and plays the sounds.
Embodiment 88. The system according to any one of Embodiments 83-87, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.
Embodiment 89. The system according to any one of Embodiments 83-88, wherein the vagal nerve stimulation module comprises one or more sense stimulation instructions for touch, and the one or more sense stimulation instructions for touch include one or more instructions for abdominal breathing.
Embodiment 90. The system according to any one of Embodiments 83-89, wherein the vagal nerve stimulation module comprises one or more sense stimulation instructions for taste, and the one or more sense stimulation instructions for taste include one or more instructions to eat food to stimulate digestive glands in the subject.
Embodiment 91. The system according to Embodiment 90, wherein the electronic device receives and display information related to the food.
Embodiment 92. The system according to any one of Embodiments 83-91, wherein the vagal nerve stimulation module comprises one or more sense stimulation instructions for smell, and the one or more sense stimulation instructions for smell include one or more instructions to stimulate digestive glands and/or to relax.
Embodiment 93. The system according to Embodiment 92, wherein the electronic device is configured to release a scent for aroma therapy.
Embodiment 94. The system according to any one of Embodiments 69-93, wherein the one or more first modules comprise the aerobic exercise module, and the aerobic exercise module comprises one or more first instructions to increase adiponectin secretion in the subject.
Embodiment 95. The system according to Embodiment 94, wherein said one or more first instructions comprise one or more instructions for walking, biking, aerobic dance and/or swimming.
Embodiment 96. The system according to Embodiment 95, wherein the wherein the electronic device displays a guide screen indicating that the subject needs rest when maximum heart rate is reached.
Embodiment 97. The system according to Embodiment 96, wherein the maximum heart rate is calculated as [{207−(0.7*age)}-basal heart rate]*0.7+basal heart Embodiment 98. The system according to Embodiment 97, wherein the basal heart rate is the subject's stable heart rate when setting up the device for the first time.
Embodiment 99. The system according to Embodiment 98, wherein when the device detects abnormal heart rate, a guide screen indicates that the subject should sufficiently rest.
Embodiment 100. The system according to Embodiment 99, wherein sufficient rest is about 10 minutes, or whenever the subject's heart rate falls below the maximum heart rate.
Embodiment 101. The system according to Embodiment 72, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.
Embodiment 102. The system according to Embodiment 101, wherein said one or more first instructions for gravity exercise sessions comprise one or more instructions for finger taps and heel taps.
Embodiment 103. The system according to Embodiment 101, wherein said one or more first instructions for anti-gravity exercise sessions comprise one or more instructions for head lifting, raising up hand, and raising up leg.
Embodiment 104. The system according to Embodiment 101, wherein said one or more first instructions for lying down exercise sessions comprise one or more instructions for hand gripping, fist clenching, and head turning.
Embodiment 105. The system according to Embodiment 101, wherein said one or more first instructions for sitting exercise sessions comprise one or more instructions for phone lifting, screen touching, raising up hands, raising up legs, hand gripping, and head turning.
Embodiment 106. The system according to Embodiment 72 or Embodiment 101, wherein voluntary skeletal muscle exercise module is scheduled for 45 days.
Embodiment 107. The system according to Embodiment 106, wherein the voluntary skeletal muscle exercise module is repeated and scheduled for a total of 90 days.
Embodiment 108. The system according to Embodiment 101 or 102, wherein one or more first instructions for gravity exercise sessions are scheduled for about 5 days.
Embodiment 109. The system according to Embodiment 101 or 103, wherein one or more first instructions for anti-gravity exercise sessions are scheduled for about 5 days Embodiment 110. The system according to Embodiment 101 or 104, wherein one or more first instructions for lying down exercise sessions are scheduled for about 20 days.
Embodiment 111. The system according to Embodiment 101 or 105, wherein one or more first instructions for sitting down exercise sessions are scheduled for about 15 days.
Embodiment 112. The system according to Embodiment 101, wherein the one or more instructions for gravity exercise sessions, anti-gravity exercise sessions, lying down exercise sessions, and sitting exercise sessions are scheduled for about 20 minutes per session each day.
Embodiment 113. The system according to Embodiment 112, wherein the electronic device displays a guide screen indicating that the subject needs rest when maximum heart rate is reached.
Embodiment 114. The system according to Embodiment 113, wherein the maximum heart rate is calculated as [{207−(0.7*age)}-basal heart rate]*0.7+basal heart rate.
Embodiment 115. The system according to Embodiment 114, wherein the basal heart rate is the subject's stable heart rate when setting up the device for the first time.
Embodiment 116. The system according to Embodiment 115, wherein when the device detects abnormal heart rate, a guide screen indicates that the subject should sufficiently rest.
Embodiment 117. The system according to Embodiment 116, wherein sufficient rest is about 10 minutes, or whenever the subject's heart rate falls below the maximum heart rate.
Embodiment 118. The system according to any one of Embodiments 69-117, wherein the one or more first modules comprise the relaxation module, and the relaxation module comprises one or more first instructions to increase adiponectin secretion in the subject.
Embodiment 119. The system according to Embodiment 118, wherein the one or more first instructions comprise one or more breathing instructions.
Embodiment 120. The system according to Embodiments 118 or 119, wherein said one or more first instructions comprise one or more sound instructions to hear relaxing sound.
Embodiment 121. The system according to Embodiment 120, wherein the device receives and plays the relaxing sound.
Embodiment 122. The system according to Embodiment 118, wherein said one or more first instructions comprise one or more meditation instructions.
Embodiment 123. The system according to Embodiment 118, wherein said one or more first instructions comprise one or more massage instructions.
Embodiment 124. The system according to any one of Embodiments 118-124, wherein said instructions are scheduled for about 5 minutes.
Embodiment 125. The system according to any one of Embodiments 69-124, wherein the subject is an early cancer patient.
Embodiment 126. The system according to any one of Embodiments 69-124, wherein the subject has a cancer mass having a diameter of 3 cm or less.
Embodiment 127. The system according to any one of Embodiments 69 and 82-124, wherein the subject is a late cancer patient, and the system excludes providing a voluntary skeletal muscle exercise module.
Embodiment 128. The system according to any one of Embodiments 69 and 82-124, wherein the subject has a cancer mass having a diameter of more than 3 cm, and the system excludes providing a voluntary skeletal muscle exercise module.
Embodiment 129. The system according to Embodiment 125 or 126, wherein the subject has moderate muscle atrophy, and the one or more first modules consists of the voluntary skeletal muscle exercise module, the vagal nerve stimulation module, the aerobic exercise module, and the relaxation module.
Embodiment 130. The system according to Embodiment 128 or 129, wherein the subject has moderate muscle atrophy, and the one or more first modules consists of the vagal nerve stimulation module, the aerobic exercise module, and the relaxation module.
Embodiment 131. The system according to any one of Embodiments 69-130, wherein the subject has moderate muscle atrophy (in spec, define the moderate muscle atrophy as still being able to walk).
Embodiment 132. The system according to any one of Embodiments 69, 82-118, and 118-128, wherein the subject has severe muscle atrophy (in spec, define the severe muscle atrophy as not being able to walk), and the system excludes providing a voluntary skeletal muscle exercise module and further excludes providing an aerobic exercise module.
Embodiment 133. The system according to Embodiment 132 wherein the one or more first modules consists of the vagal nerve stimulation module and the relaxation module.
Embodiment 134. The system according to any one of Embodiments 69-133, wherein the digital application transmits data to a server, and wherein the server receives the one or more second instructions from an external reviewer.
Embodiment 135. The system according to any one of Embodiments 69-134, wherein the external reviewer comprises a health professional.
Embodiment 136. The system according to any one of Embodiments 69-134, wherein the external reviewer comprises an artificial intelligence (AI).
Embodiment 137. The system according to any one of Embodiments 69-136, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.
Embodiment 138. The system according to any one of Embodiments 69-137, wherein the sensor comprises a touch sensor, and the subject provides the adherence information to the electronic device using the touch sensor.
Embodiment 139. The system according to any one of Embodiment 69-138, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.
Embodiment 140. The system of Embodiment 139, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.
Embodiment 141. The system of Embodiment 140, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.
Embodiment 142. The system of any one of Embodiments 139-141, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
Embodiment 143. The system of any one of Embodiments 139-143, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
Embodiment 144. The system of Embodiment 69-143, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.
Embodiment 145. The system of Embodiment 144, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.
Embodiment 146. The system of Embodiment 144 or 145, wherein the one or more options comprise the viewing or editing the de-identified information of the subject, and the de-identified information of the subject comprises one or more selected from the group consisting of an identification number for the subject, and the healthcare provider for the subject.
Embodiment 147. The system of any one of Embodiments 144-146, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
Embodiment 148. The system of any one of Embodiments 139-147, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
Embodiment 149. The system of Embodiment 69-148, wherein the digital application further comprises a push alarm for one or more of reminding the subject complete a digital therapeutic module.
Embodiment 150. The system of Embodiment 69-149, wherein the digital apparatus comprises: a digital instruction generation unit configured to generate digital therapeutic modules for treating cancer cachexia, generate digital instructions based on the digital therapeutic modules, and provide the digital instructions to the subject; and an outcome collection unit configured to collect the subject's execution outcomes of the digital instructions.
Embodiment 151. The system of Embodiment 69-150, wherein the digital instruction generation unit generates the digital therapeutic modules based on biochemical factors related to the cancer cachexia onset.
Embodiment 152. The system of Embodiment 151, wherein the biochemical factors comprise insulin-like growth factor 1 (IGF1) and hypoxia-inducible factor 1 (HIF1).
Embodiment 153. The system of Embodiment 69-152, wherein the digital instruction generation unit generates the digital therapeutic modules based on the inputs from the healthcare provider.
Embodiment 154. The system of Embodiment 69-153, wherein the digital instruction generation unit generates the digital therapeutic modules based on information received from the subject.
Embodiment 155. The system of Embodiment 154, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.
Embodiment 156. The system of Embodiment 69-155, wherein the digital instruction generation unit generates the digital therapeutic modules matching to imaginary parameters which correspond to the mechanism of action in and the therapeutic hypothesis for the cancer cachexia.
Embodiment 157. The system of Embodiment 156, wherein the imaginary parameters are deduced in relation to the subject's environment, behaviors, emotions, and cognition.
Embodiment 158. The system of Embodiment 69-157, wherein the outcome collection unit collects the execution outcomes of the digital instructions by monitoring the subject's adherence to the digital instructions or allowing the subject to directly input the subject's adherence to the digital instructions.
Embodiment 159. The system of Embodiment 69-158, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.
Embodiment 160. A computing system for treating cancer cachexia in a subject in need thereof, comprising: a display configured to provide, to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; a sensor configured to sense adherence by the subject to the instructions of the one or more first modules; a transmitter configured to transmit adherence information, based on the adherence, to a server; and a receiver configured to receive, from the server, one or more second instructions based on the adherence information, wherein the display is further configured to provide, to the subject, one or more second modules selected from the group consisting of a voluntary skeletal muscle exercise module, a vagal nerve stimulation module, an aerobic exercise module, and a relaxation module, each of the one or more second modules comprising the one or more second instructions.
Embodiment 161. The computing system of Embodiment 160, wherein the digital application for treating cancer cachexia instructs a processor of the digital apparatus to execute operations comprising: generating digital therapeutic modules for treating cancer cachexia based on a mechanism of action in and a therapeutic hypothesis for the cancer cachexia.
Embodiment 162. The computing system of Embodiment 161, wherein the generating of the digital therapeutic modules comprises generating the digital therapeutic modules based on biochemical factors related to the cancer cachexia.
Embodiment 163. The computing system of Embodiment 162, wherein the one or more first modules comprise the voluntary skeletal muscle exercise module, and the voluntary skeletal muscle exercise module comprises one or more first instructions to increase MGF/IGF-1 secretion in the subject.
Embodiment 164. The computing system of Embodiment 163, wherein the voluntary skeletal muscle exercise module comprises a screen touching exercise instruction to touch a randomly generated displayed animation on the electronic device.
Embodiment 165. The computing system of Embodiment 163, wherein the voluntary skeletal muscle exercise module comprises a head lifting exercise instruction to lift and position a head of the subject at a predetermined angle from the reference plane.
Embodiment 166. The computing system of Embodiment 163, wherein the voluntary skeletal muscle exercise module comprises a head turning exercise instruction to turn a head of the subject clockwise and/or counterclockwise at a predetermined angle from the reference plane.
Embodiment 167. The computing system of Embodiment 163, wherein the voluntary skeletal muscle exercise module comprises a hand gripping exercise instruction to grip a fist of the subject at a rate matching a displayed animation on the electronic device.
Embodiment 168. The computing system of Embodiment 163, wherein the voluntary skeletal muscle exercise module comprises an arm shaking exercise instruction to move an object displayed on the electronic device across the screen with an index finger of the subject.
Embodiment 169. The computing system of Embodiment 163, wherein the voluntary skeletal muscle exercise module comprises a leg lifting exercise instruct to lift their legs as measured by a change in a background area captured by the electronic device.
Embodiment 170. The computing system of Embodiment 163, wherein said one or more first instructions comprise instructions for aerobic, resistance, and/or concurrent exercise.
Embodiment 171. The computing system of any one of Embodiments 160-170, wherein the subject inputs health information into the electronic device.
Embodiment 172. The computing system of Embodiment 171, wherein the health information comprises of the subject's sleep schedule, blood sugar levels, protein, amino acid, anti-inflammatory health food intake information, appetite stimulant consumption information, and albumin levels.
Embodiment 173. The computing system of any one of Embodiments 160-172, wherein the one or more first modules comprise the vagal nerve stimulation module, and the vagal nerve stimulation module comprises one or more first instructions to reduce inflammation in the subject.
Embodiment 174. The computing system of any one of Embodiments 160-173, wherein the one or more first modules comprise the vagal nerve stimulation module, and the vagal nerve stimulation module comprises at least one instruction selected from the group consisting of sense stimulation instructions for sight, sound, touch, taste, and smell.
Embodiment 175. The computing system of Embodiment 174, wherein the vagal nerve stimulation module comprises one or more sense stimulation instructions for sight, and the one or more sense stimulation instructions for sight include one or more instructions to view one or more figures (define in spec to include specific photos) to stimulate autonomic nervous system.
Embodiment 176. The computing system of Embodiment 175, wherein the electronic device receives and displays the figures.
Embodiment 177. The computing system of any one of Embodiments 173-175, wherein the vagal nerve stimulation module comprises one or more sense stimulation instructions for sound, and the one or more sense stimulation instructions for sound include one or more instructions to hear one or more sounds to cause horror or relaxation.
Embodiment 178. The computing system of Embodiment 177, wherein the electronic device receives and plays the sounds.
Embodiment 179. The computing system of any one of Embodiments 173-178, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.
Embodiment 180. The computing system of any one of Embodiments 173-179, wherein the vagal nerve stimulation module comprises one or more sense stimulation instructions for touch, and the one or more sense stimulation instructions for touch include one or more instructions for abdominal breathing.
Embodiment 181. The computing system of any one of Embodiments 173-180, wherein the vagal nerve stimulation module comprises one or more sense stimulation instructions for taste, and the one or more sense stimulation instructions for taste include one or more instructions to eat food to stimulate digestive glands in the subject.
Embodiment 182. The computing system of Embodiment 181, wherein the electronic device receives and display information related to the food.
The computing system of any one of Embodiments 173-182, wherein the vagal nerve stimulation module comprises one or more sense stimulation instructions for smell, and the one or more sense stimulation instructions for smell include one or more instructions to stimulate digestive glands and/or to relax.
Embodiment 183. The computing system of Embodiment 183, wherein the electronic device is configured to release a scent for aroma therapy.
Embodiment 184. The computing system of any one of Embodiments 160-184, wherein the one or more first modules comprise the aerobic exercise module, and the aerobic exercise module comprises one or more first instructions to increase adiponectin secretion in the subject.
Embodiment 185. The computing system of Embodiment 185, wherein said one or more first instructions comprise one or more instructions for walking, biking, aerobic dance and/or swimming.
Embodiment 186. The computing system of Embodiment 186, wherein the wherein the electronic device displays a guide screen indicating that the subject needs rest when maximum heart rate is reached.
Embodiment 187. The computing system of Embodiment 187, wherein the maximum heart rate is calculated as [{207−(0.7*age)}-basal heart rate]*0.7+basal heart Embodiment 188. The computing system of Embodiment 188, wherein the basal heart rate is the subject's stable heart rate when setting up the device for the first time.
Embodiment 189. The computing system of Embodiment 189, wherein when the device detects abnormal heart rate, a guide screen indicates that the subject should sufficiently rest.
Embodiment 190. The computing system of Embodiment 190, wherein sufficient rest is about 10 minutes, or whenever the subject's heart rate falls below the maximum heart rate.
Embodiment 191. The computing system of Embodiment 163, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.
Embodiment 192. The computing system of Embodiment 192, wherein said one or more first instructions for gravity exercise sessions comprise one or more instructions for finger taps and heel taps.
Embodiment 193. The computing system of Embodiment 192, wherein said one or more first instructions for anti-gravity exercise sessions comprise one or more instructions for head lifting, raising up hand, and raising up leg.
Embodiment 194. The computing system of Embodiment 192, wherein said one or more first instructions for lying down exercise sessions comprise one or more instructions for hand gripping, fist clenching, and head turning.
Embodiment 195. The computing system of Embodiment 192, wherein said one or more first instructions for sitting exercise sessions comprise one or more instructions for phone lifting, screen touching, raising up hands, raising up legs, hand gripping, and head turning.
Embodiment 196. The computing system of Embodiment 163 or Embodiment 192, wherein voluntary skeletal muscle exercise module is scheduled for 45 days.
Embodiment 197. The computing system of Embodiment 195, wherein the voluntary skeletal muscle exercise module is repeated and scheduled for a total of 90 days.
Embodiment 198. The computing system of Embodiment 192 or 193, wherein one or more first instructions for gravity exercise sessions are scheduled for about 5 days.
Embodiment 199. The computing system of Embodiment 192 or 194, wherein one or more first instructions for anti-gravity exercise sessions are scheduled for about 5 days Embodiment 200. The computing system of Embodiment 192 or 195, wherein one or more first instructions for lying down exercise sessions are scheduled for about 20 days.
Embodiment 201. The computing system of Embodiment 192 or 196, wherein one or more first instructions for sitting down exercise sessions are scheduled for about 15 days.
Embodiment 202. The computing system of Embodiment 192, wherein the one or more instructions for gravity exercise sessions, anti-gravity exercise sessions, lying down exercise sessions, and sitting exercise sessions are scheduled for about 20 minutes per session each day.
Embodiment 203. The computing system of Embodiment 203, wherein the electronic device displays a guide screen indicating that the subject needs rest when maximum heart rate is reached.
Embodiment 204. The computing system of Embodiment 204, wherein the maximum heart rate may be calculated as [{207−(0.7*age)}-basal heart rate]*0.7+basal heart rate.
Embodiment 205. The computing system of Embodiment 205, wherein the basal heart rate is the subject's stable heart rate when setting up the device for the first time.
Embodiment 206. The computing system of Embodiment 206, wherein when the device detects abnormal heart rate, a guide screen indicates that the subject should sufficiently rest.
Embodiment 207. The computing system of Embodiment 207, wherein sufficient rest is about 10 minutes, or whenever the subject's heart rate falls below the maximum heart rate.
Embodiment 208. The computing system of any one of Embodiments 160-208, wherein the one or more first modules comprise the relaxation module, and the relaxation module comprises one or more first instructions to increase adiponectin secretion in the subject.
Embodiment 209. The computing system of Embodiment 209, wherein the one or more first instructions comprise one or more breathing instructions.
Embodiment 210. The computing system of Embodiment 209 or 210, wherein said one or more first instructions comprise one or more sound instructions to hear relaxing sound.
Embodiment 211. The computing system of Embodiment 211, wherein the device receives and plays the relaxing sound.
Embodiment 212. The computing system of Embodiment 209, wherein said one or more first instructions comprise one or more meditation instructions.
Embodiment 213. The computing system of Embodiment 209, wherein said one or more first instructions comprise one or more massage instructions.
Embodiment 214. The computing system of any one of Embodiments 209-214, wherein said instructions are scheduled for about 5 minutes.
Embodiment 215. The computing system of any one of Embodiments 160-214, wherein the subject is an early cancer patient.
Embodiment 216. The computing system of any one of Embodiments 160-214, wherein the subject has a cancer mass having a diameter of 3 cm or less.
Embodiment 217. The computing system of any one of Embodiments 160 and 173-215, wherein the subject is a late cancer patient, and the system excludes providing a voluntary skeletal muscle exercise module.
Embodiment 218. The computing system of any one of Embodiments 160 and 173-215, wherein the subject has a cancer mass having a diameter of more than 3 cm, and the system excludes providing a voluntary skeletal muscle exercise module.
Embodiment 219. The computing system of Embodiment 216 or 217, wherein the subject has moderate muscle atrophy, and the one or more first modules consists of the voluntary skeletal muscle exercise module, the vagal nerve stimulation module, the aerobic exercise module, and the relaxation module.
Embodiment 220. The computing system of Embodiment 219 or 220, wherein the subject has moderate muscle atrophy, and the one or more first modules consists of the vagal nerve stimulation module, the aerobic exercise module, and the relaxation module.
Embodiment 221. The computing system of any one of Embodiments 160-221, wherein the subject has moderate muscle atrophy (in spec, define the moderate muscle atrophy as still being able to walk).
Embodiment 222. The computing system of any one of Embodiments 160, 173-209, and 209-219, wherein the subject has severe muscle atrophy (in spec, define the severe muscle atrophy as not being able to walk), and the system excludes providing a voluntary skeletal muscle exercise module and further excludes providing an aerobic exercise module.
Embodiment 223. The computing system of Embodiment 223, wherein the one or more first modules consists of the vagal nerve stimulation module and the relaxation module.
Embodiment 224. The computing system of any one of Embodiments 160-224, wherein the digital application transmits data to a server, and wherein the server receives the one or more second instructions from an external reviewer.
Embodiment 225. The computing system of any one of Embodiments 160-225, wherein the external reviewer comprises a health professional.
Embodiment 226. The computing system of any one of Embodiments 160-225, wherein the external reviewer comprises an artificial intelligence (AI).
Embodiment 227. The computing system of any one of Embodiments 160-227, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.
Embodiment 228. The computing system of any one of Embodiments 160-228, wherein the sensor comprises a touch sensor, and the subject provides the adherence information to the electronic device using the touch sensor.
Embodiment 229. The computing system of Embodiment 160-229, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.
Embodiment 230. The computing system of Embodiment 230, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.
Embodiment 231. The computing system of Embodiment 231, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.
Embodiment 232. The computing system of Embodiment 232, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
Embodiment 233. The computing system of Embodiment 232, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
Embodiment 234. The computing system of Embodiment 160-234, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.
Embodiment 235. The computing system of Embodiment 235, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.
Embodiment 236. The computing system of Embodiment 235, wherein the one or more options comprise the viewing or editing the de-identified information of the subject, and the de-identified information of the subject comprises one or more selected from the group consisting of an identification number for the subject, and the healthcare provider for the subject.
Embodiment 237. The computing system of Embodiment 235, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
Embodiment 238. The computing system of Embodiment 235, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
Embodiment 239. The computing system of Embodiment 160-239, wherein the digital application further comprises a push alarm for one or more of reminding the subject complete a digital therapeutic module.
Embodiment 240. The computing system of Embodiment 160-240, wherein the digital apparatus comprises: a digital instruction generation unit configured to generate digital therapeutic modules for treating cancer cachexia, generate digital instructions based on the digital therapeutic modules, and provide the digital instructions to the subject; and an outcome collection unit configured to collect the subject's execution outcomes of the digital instructions.
Embodiment 241. The computing system of Embodiment 160-241, wherein the digital instruction generation unit generates the digital therapeutic modules based on biochemical factors related to the cancer cachexia onset.
Embodiment 242. The computing system of Embodiment 242, wherein the biochemical factors comprise insulin-like growth factor 1 (IGF1) and hypoxia-inducible factor 1 (HIF1).
Embodiment 243. The computing system of Embodiment 160-243, wherein the digital instruction generation unit generates the digital therapeutic modules based on the inputs from the healthcare provider.
Embodiment 244. The computing system of Embodiment 160-244, wherein the digital instruction generation unit generates the digital therapeutic modules based on information received from the subject.
Embodiment 245. The computing system of Embodiment 245, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.
Embodiment 246. The computing system of Embodiment 160-246, wherein the digital instruction generation unit generates the digital therapeutic modules matching to imaginary parameters which correspond to the mechanism of action in and the therapeutic hypothesis for the cancer cachexia.
Embodiment 247. The computing system of Embodiment 160, wherein the imaginary parameters are deduced in relation to the subject's environment, behaviors, emotions, and cognition.
Embodiment 248. The computing system of Embodiment 160-248, wherein the outcome collection unit collects the execution outcomes of the digital instructions by monitoring the subject's adherence to the digital instructions or allowing the subject to directly input the subject's adherence to the digital instructions.
Embodiment 249. The computing system of Embodiment 160-249, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.
Embodiment 250. A non-transitory computer readable medium having stored thereon software instructions for treating cancer cachexia in a subject in need thereof that, when executed by a processor, cause the processor to: display, by an electronic device to the subject, one or more first modules selected from the group consisting of a voluntary skeletal muscle exercise module, a vagal nerve stimulation module, an aerobic exercise module, and a relaxation module, each of the one or more first modules comprising instructions for the subject to follow; sense, by a sensor in the electronic device, adherence by the subject to the instructions of the one or more first modules; transmit, by the electronic device, adherence information, based on the adherence, to a server; receive, from the server, one or more second instructions based on the adherence information; and display, to the subject, one or more second modules selected from the group consisting of a voluntary skeletal muscle exercise module, a vagal nerve stimulation module, an aerobic exercise module, and a relaxation module, the one or more second modules comprising the one or more second instructions.
Embodiment 251. The non-transitory computer readable medium of Embodiment 251, wherein the digital application for treating cancer cachexia instructs a processor of the digital apparatus to execute operations comprising: generating digital therapeutic modules for treating cancer cachexia based on a mechanism of action in and a therapeutic hypothesis for the cancer cachexia.
Embodiment 252. The non-transitory computer readable medium of Embodiment 252, wherein the generating of the digital therapeutic modules comprises generating the digital therapeutic modules based on biochemical factors related to the cancer cachexia.
Embodiment 253. The non-transitory computer readable medium of Embodiment 253, wherein the one or more first modules comprise the voluntary skeletal muscle exercise module, and the voluntary skeletal muscle exercise module comprises one or more first instructions to increase MGF/IGF-1 secretion in the subject.
Embodiment 254. The non-transitory computer readable medium of Embodiment 254, wherein the voluntary skeletal muscle exercise module comprises a screen touching exercise instruction to touch a randomly generated displayed animation on the electronic device.
Embodiment 255. The non-transitory computer readable medium of Embodiment 254, wherein the voluntary skeletal muscle exercise module comprises a head lifting exercise instruction to lift and position a head of the subject at a predetermined angle from the reference plane.
Embodiment 256. The non-transitory computer readable medium of Embodiment 254, wherein the voluntary skeletal muscle exercise module comprises a head turning exercise instruction to turn a head of the subject clockwise and/or counterclockwise at a predetermined angle from the reference plane.
Embodiment 257. The non-transitory computer readable medium of Embodiment 254, wherein the voluntary skeletal muscle exercise module comprises a hand gripping exercise instruction to grip a fist of the subject at a rate matching a displayed animation on the electronic device.
Embodiment 258. The non-transitory computer readable medium of Embodiment 254, wherein the voluntary skeletal muscle exercise module comprises an arm shaking exercise instruction to move an object displayed on the electronic device across the screen with an index finger of the subject.
Embodiment 259. The non-transitory computer readable medium of Embodiment 254, wherein the voluntary skeletal muscle exercise module comprises a leg lifting exercise instruct to lift their legs as measured by a change in a background area captured by the electronic device.
Embodiment 260. The non-transitory computer readable medium of Embodiment 254, wherein said one or more first instructions comprise instructions for aerobic, resistance, and/or concurrent exercise.
Embodiment 261. The non-transitory computer readable medium of any one of Embodiments 251-261, wherein the subject inputs health information into the electronic device.
Embodiment 262. The non-transitory computer readable medium of Embodiment 262, wherein the health information comprises of the subject's sleep schedule, blood sugar levels, protein, amino acid, anti-inflammatory health food intake information, appetite stimulant consumption information, and albumin levels.
Embodiment 263. The non-transitory computer readable medium of any one of Embodiments 251-263, wherein the one or more first modules comprise the vagal nerve stimulation module, and the vagal nerve stimulation module comprises one or more first instructions to reduce inflammation in the subject.
Embodiment 264. The non-transitory computer readable medium of any one of Embodiments 251-264, wherein the one or more first modules comprise the vagal nerve stimulation module, and the vagal nerve stimulation module comprises at least one instruction selected from the group consisting of sense stimulation instructions for sight, sound, touch, taste, and smell.
Embodiment 265. The non-transitory computer readable medium of Embodiment 265, wherein the vagal nerve stimulation module comprises one or more sense stimulation instructions for sight, and the one or more sense stimulation instructions for sight include one or more instructions to view one or more figures (define in spec to include specific photos) to stimulate autonomic nervous system.
Embodiment 266. The non-transitory computer readable medium of Embodiment 266, wherein the electronic device receives and displays the figures.
Embodiment 267. The non-transitory computer readable medium of any one of Embodiments 265-267, wherein the vagal nerve stimulation module comprises one or more sense stimulation instructions for sound, and the one or more sense stimulation instructions for sound include one or more instructions to hear one or more sounds to cause horror or relaxation.
Embodiment 268. The non-transitory computer readable medium of Embodiment 268, wherein the electronic device receives and plays the sounds.
Embodiment 269. The non-transitory computer readable medium of any one of Embodiments 265-269, wherein the vagal nerve stimulation module comprises one or more sense stimulation instructions for touch, and the one or more sense stimulation instructions for touch include one or more instructions for abdominal breathing, controlling rate of breathing, cold massage, coughing, and skin massage.
Embodiment 270. The non-transitory computer readable medium of any one of Embodiments 265-270, wherein the vagal nerve stimulation module comprises one or more sense stimulation instructions for touch, and the one or more sense stimulation instructions for touch include one or more instructions for abdominal breathing.
Embodiment 271. The non-transitory computer readable medium of any one of Embodiments 265-271, wherein the vagal nerve stimulation module comprises one or more sense stimulation instructions for taste, and the one or more sense stimulation instructions for taste include one or more instructions to eat food to stimulate digestive glands in the subject.
Embodiment 272. The non-transitory computer readable medium of Embodiment 272, wherein the electronic device receives and display information related to the food.
Embodiment 273. The non-transitory computer readable medium of any one of Embodiments 265-273, wherein the vagal nerve stimulation module comprises one or more sense stimulation instructions for smell, and the one or more sense stimulation instructions for smell include one or more instructions to stimulate digestive glands and/or to relax.
Embodiment 274. The non-transitory computer readable medium of Embodiment 274, wherein the electronic device is configured to release a scent for aroma therapy.
Embodiment 275. The non-transitory computer readable medium of any one of Embodiments 251-275, wherein the one or more first modules comprise the aerobic exercise module, and the aerobic exercise module comprises one or more first instructions to increase adiponectin secretion in the subject.
Embodiment 276. The non-transitory computer readable medium of Embodiment 276, wherein said one or more first instructions comprise one or more instructions for walking, biking, aerobic dance and/or swimming.
Embodiment 277. The non-transitory computer readable medium of Embodiment 277, wherein the wherein the electronic device displays a guide screen indicating that the subject needs rest when maximum heart rate is reached.
Embodiment 278. The non-transitory computer readable medium of Embodiment 278, wherein the maximum heart rate is calculated as [{207−(0.7*age)}-basal heart rate]*0.7+basal heart Embodiment 279. The non-transitory computer readable medium of Embodiment 279, wherein the basal heart rate is the subject's stable heart rate when setting up the device for the first time.
Embodiment 280. The non-transitory computer readable medium of Embodiment 280, wherein when the device detects abnormal heart rate, a guide screen indicates that the subject should sufficiently rest.
Embodiment 281. The non-transitory computer readable medium of Embodiment 281, wherein sufficient rest is about 10 minutes, or whenever the subject's heart rate falls below the maximum heart rate.
Embodiment 282. The non-transitory computer readable medium of Embodiment 254, wherein the one or more first modules comprise the voluntary skeletal muscle exercise module, and the voluntary skeletal muscle exercise module compromise one or more first instructions for gravity exercise sessions, anti-gravity exercise sessions, lying down exercise sessions, and sitting exercise sessions.
Embodiment 283. The non-transitory computer readable medium of Embodiment 283, wherein said one or more first instructions for gravity exercise sessions comprise one or more instructions for finger taps and heel taps.
Embodiment 284. The non-transitory computer readable medium of Embodiment 283, wherein said one or more first instructions for anti-gravity exercise sessions comprise one or more instructions for head lifting, raising up hand, and raising up leg.
Embodiment 285. The non-transitory computer readable medium of Embodiment 283, wherein said one or more first instructions for lying down exercise sessions comprise one or more instructions for hand gripping, fist clenching, and head turning.
Embodiment 286. The non-transitory computer readable medium of Embodiment 283, wherein said one or more first instructions for sitting exercise sessions comprise one or more instructions for phone lifting, screen touching, raising up hands, raising up legs, hand gripping, and head turning.
Embodiment 287. The non-transitory computer readable medium of Embodiment 251 or Embodiment 283, wherein voluntary skeletal muscle exercise module is scheduled for 45 days.
Embodiment 288. The non-transitory computer readable medium of Embodiment 288, wherein the voluntary skeletal muscle exercise module is repeated and scheduled for a total of 90 days.
Embodiment 289. The non-transitory computer readable medium of Embodiment 283 or 284, wherein one or more first instructions for gravity exercise sessions are scheduled for about 5 days.
Embodiment 290. The non-transitory computer readable medium of Embodiment 283 or 285, wherein one or more first instructions for anti-gravity exercise sessions are scheduled for about 5 days Embodiment 291. The non-transitory computer readable medium of Embodiment 283 or 286, wherein one or more first instructions for lying down exercise sessions are scheduled for about 20 days.
Embodiment 292. The non-transitory computer readable medium of Embodiment 283 or 287, wherein one or more first instructions for sitting down exercise sessions are scheduled for about 15 days.
Embodiment 293. The non-transitory computer readable medium of Embodiment 283, wherein the one or more instructions for gravity exercise sessions, anti-gravity exercise sessions, lying down exercise sessions, and sitting exercise sessions are scheduled for about 20 minutes per session each day.
Embodiment 294. The non-transitory computer readable medium of Embodiment 294, wherein the electronic device displays a guide screen indicating that the subject needs rest when maximum heart rate is reached.
Embodiment 295. The non-transitory computer readable medium of Embodiment 295, wherein the maximum heart rate is calculated as [{207−(0.7*age)}-basal heart rate]*0.7+basal heart rate.
Embodiment 296. The non-transitory computer readable medium of Embodiment 296, wherein the basal heart rate is the subject's stable heart rate when setting up the device for the first time.
Embodiment 297. The non-transitory computer readable medium of Embodiment 297, wherein when the device detects abnormal heart rate, a guide screen indicates that the subject should sufficiently rest.
Embodiment 298. The non-transitory computer readable medium of according to Embodiment 298, wherein sufficient rest is about 10 minutes, or whenever the subject's heart rate falls below the maximum heart rate.
Embodiment 299. The non-transitory computer readable medium of any one of Embodiments 251-299, wherein the one or more first modules comprise the relaxation module, and the relaxation module comprises one or more first instructions to increase adiponectin secretion in the subject.
Embodiment 300. The non-transitory computer readable medium of Embodiment 300, wherein the one or more first instructions comprise one or more breathing instructions.
Embodiment 301. The non-transitory computer readable medium of Embodiment 300 or 301, wherein said one or more first instructions comprise one or more sound instructions to hear relaxing sound.
Embodiment 302. The non-transitory computer readable medium of Embodiment 302, wherein the device receives and plays the relaxing sound.
Embodiment 303. The non-transitory computer readable medium of Embodiment 300, wherein said one or more first instructions comprise one or more meditation instructions.
Embodiment 304. The non-transitory computer readable medium of Embodiment 300, wherein said one or more first instructions comprise one or more massage instructions.
Embodiment 305. The non-transitory computer readable medium of any one of Embodiments 300-305, wherein said instructions are scheduled for about 5 minutes.
Embodiment 306. The non-transitory computer readable medium of any one of Embodiments 251-306, wherein the subject is an early cancer patient.
Embodiment 307. The non-transitory computer readable medium of any one of Embodiments 251-306, wherein the subject has a cancer mass having a diameter of 3 cm or less.
Embodiment 308. The non-transitory computer readable medium of any one of Embodiments 251 and 271-306, wherein the subject is a late cancer patient, and the system excludes providing a voluntary skeletal muscle exercise module.
Embodiment 309. The non-transitory computer readable medium of any one of Embodiments 251 and 271-306, wherein the subject has a cancer mass having a diameter of more than 3 cm, and the system excludes providing a voluntary skeletal muscle exercise module.
Embodiment 310. The non-transitory computer readable medium of Embodiment 307 or 308, wherein the subject has moderate muscle atrophy, and the one or more first modules consists of the voluntary skeletal muscle exercise module, the vagal nerve stimulation module, the aerobic exercise module, and the relaxation module.
Embodiment 311. The non-transitory computer readable medium of Embodiment 309 or 310, wherein the subject has moderate muscle atrophy, and the one or more first modules consists of the vagal nerve stimulation module, the aerobic exercise module, and the relaxation module.
Embodiment 312. The non-transitory computer readable medium of any one of Embodiments 251-312, wherein the subject has moderate muscle atrophy (in spec, define the moderate muscle atrophy as still being able to walk).
Embodiment 313. The non-transitory computer readable medium of any one of Embodiments 251, 271-300, and 300-310 wherein the subject has severe muscle atrophy (in spec, define the severe muscle atrophy as not being able to walk), and the system excludes providing a voluntary skeletal muscle exercise module and further excludes providing an aerobic exercise module.
Embodiment 314. The non-transitory computer readable medium of Embodiment 314, wherein the one or more first modules consists of the vagal nerve stimulation module and the relaxation module.
Embodiment 315. The non-transitory computer readable medium of any one of Embodiments 251-315, wherein the digital application transmits data to a server, and wherein the server receives the one or more second instructions from an external reviewer.
Embodiment 316. The non-transitory computer readable medium of any one of Embodiments 251-316, wherein the external reviewer comprises a health professional.
Embodiment 317. The non-transitory computer readable medium of any one of Embodiments 251-317, wherein the external reviewer comprises an artificial intelligence (AI).
Embodiment 318. The non-transitory computer readable medium of any one of Embodiments 251-318, wherein the sensor comprises one or more of: a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.
Embodiment 319. The non-transitory computer readable medium of any one of Embodiments 251-319, wherein the sensor comprises a touch sensor, and the subject provides the adherence information to the electronic device using the touch sensor.
Embodiment 320. The non-transitory computer readable medium of Embodiment 251-320, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.
Embodiment 321. The non-transitory computer readable medium of Embodiment 321, wherein the one or more options comprise the viewing or editing personal information for the subject, and the personal information comprises one or more selected from the group consisting of an identification number for the subject, a name of the subject, a date of birth of the subject, an email of the subject, an email of the guardian of the subject, a contact phone number for the subject, a prescription for the subject, and one or more notes made by the healthcare provider about the subject.
Embodiment 322. The non-transitory computer readable medium of Embodiment 322, wherein the personal information comprises the prescription for the subject, and the prescription for the subject comprises one or more selected from the group consisting of a prescription identification number, a prescription type, a start date, a duration, a completion date, a number of scheduled or prescribed digital therapeutic modules to be performed by the subject, and a number of scheduled or prescribed digital therapeutic modules to be performed by the subject per day.
Embodiment 323. The non-transitory computer readable medium of any one of Embodiments 321-323, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
Embodiment 324. The non-transitory computer readable medium of any one of Embodiments 321-324, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
Embodiment 325. The non-transitory computer readable medium of Embodiment 251-325, wherein the one or more options provided to the administrator of the system are selected from the group consisting of adding or removing the healthcare provider, viewing or editing personal information for the healthcare provider, viewing or editing de-identified information of the subject, viewing adherence information for the subject, viewing a result of the subject for one or more at least partially completed digital therapeutic modules, and communicating with the healthcare provider.
Embodiment 326. The non-transitory computer readable medium of Embodiment 326, wherein the one or more options comprise the viewing or editing the personal information, and the personal information of the healthcare provider comprises one or more selected from the group consisting of an identification number for the healthcare provider, a name of the healthcare provider, an email of the healthcare provider, and a contact phone number for the healthcare provider.
Embodiment 327. The non-transitory computer readable medium of Embodiment 326 or 327, wherein the one or more options comprise the viewing or editing the de-identified information of the subject, and the de-identified information of the subject comprises one or more selected from the group consisting of an identification number for the subject, and the healthcare provider for the subject.
Embodiment 328. The non-transitory computer readable medium of any one of Embodiments 326-328, wherein the one or more options comprise the viewing the adherence information for the subject, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.
Embodiment 329. The non-transitory computer readable medium of any one of Embodiments 326-329, wherein the one or more options comprise the viewing the result of the subject, and the result of the subject for one or more at least partially completed digital therapeutic modules comprises one or more selected from the group consisting of a time at which the subject started a scheduled or prescribed digital therapeutic module, a time at which the subject ended a scheduled or prescribed digital therapeutic module, an indicator of whether the scheduled or prescribed digital therapeutic module was fully or partially completed, and an exercise intensity (EI).
Embodiment 330. The non-transitory computer readable medium of Embodiment 251-330, wherein the digital application further comprises a push alarm for one or more of reminding the subject complete a digital therapeutic module.
Embodiment 331. The non-transitory computer readable medium of Embodiment 251-331, wherein the digital apparatus comprises: a digital instruction generation unit configured to generate digital therapeutic modules for treating cancer cachexia, generate digital instructions based on the digital therapeutic modules, and provide the digital instructions to the subject; and an outcome collection unit configured to collect the subject's execution outcomes of the digital instructions.
Embodiment 332. The non-transitory computer readable medium of Embodiment 251-332, wherein the digital instruction generation unit generates the digital therapeutic modules based on biochemical factors related to the cancer cachexia onset.
Embodiment 333. The non-transitory computer readable medium of Embodiment 333, wherein the biochemical factors comprise insulin-like growth factor 1 (IGF1) and hypoxia-inducible factor 1 (HIF1).
Embodiment 334. The non-transitory computer readable medium of Embodiment 251-334, wherein the digital instruction generation unit generates the digital therapeutic modules based on the inputs from the healthcare provider.
Embodiment 335. The non-transitory computer readable medium of Embodiment 251-335, wherein the digital instruction generation unit generates the digital therapeutic modules based on information received from the subject.
Embodiment 336. The non-transitory computer readable medium of Embodiment 336, wherein the information is received from the subject comprises at least one of basal factors, medical information, and digital therapeutics literacy of the subject, the basal factors including the subject's activity, heart rate, sleep, and diet (including nutrition and calories), the medical information including the subject's electronic medical record (EMR), family history, genetic vulnerability, and genetic susceptibility, and the digital therapeutics literacy including the subject's accessibility, and technology adoption to the digital therapeutics and the apparatus.
Embodiment 337. The non-transitory computer readable medium of Embodiment 251-337, wherein the digital instruction generation unit generates the digital therapeutic modules matching to imaginary parameters which correspond to the mechanism of action in and the therapeutic hypothesis for the cancer cachexia.
Embodiment 338. The non-transitory computer readable medium of Embodiment 338, wherein the imaginary parameters are deduced in relation to the subject's environment, behaviors, emotions, and cognition.
Embodiment 339. The non-transitory computer readable medium of Embodiment 251-339, wherein the outcome collection unit collects the execution outcomes of the digital instructions by monitoring the subject's adherence to the digital instructions or allowing the subject to directly input the subject's adherence to the digital instructions.
Embodiment 340. The non-transitory computer readable medium of Embodiment 251-340, wherein the generation of the digital instructions at the digital instruction generation unit and the collection of the subject's execution outcomes of the digital instructions at the outcome collection unit are repeatedly executed several times with multiple feedback loops, and the digital instruction generation unit generates the subject's digital instructions for this cycle based on the subject's digital instructions in the previous cycle and the execution outcome data on the subject's digital instructions in the previous cycle collected at the outcome collection unit.

What is claimed is:

1. A method of treating cancer cachexia in a subject in need thereof, the method comprising:
providing, by an electronic device to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow; and
providing, by the electronic device to the subject, one or more second modules selected from the group consisting of a voluntary skeletal muscle exercise module, a vagal nerve stimulation module, an aerobic exercise module, and a relaxation module, the one or more second modules comprising the one or more second instructions,
wherein the electronic device comprises a sensor sensing adherence by the subject to the first instructions of the one or more first modules, transmits adherence information, based on the adherence, to a server, and receives one or more second instructions from the server based on the adherence information.

2. The method according to claim 1, wherein the one or more first modules comprise the voluntary skeletal muscle exercise module, and the voluntary skeletal muscle exercise module comprises one or more instructions to increase MGF/IGF-1 secretion in the subject.

3. The method according to claim 2, wherein the voluntary skeletal muscle exercise module comprises a screen touching exercise instruction to touch a randomly generated displayed animation on the electronic device.

4. The method according to claim 2, wherein the voluntary skeletal muscle exercise module comprises a head lifting exercise instruction to lift and position a head of the subject at a predetermined angle from the reference plane.

5. The method according to claim 2, wherein the voluntary skeletal muscle exercise module comprises a head turning exercise instruction to turn a head of the subject clockwise and/or counterclockwise at a predetermined angle from the reference plane.

6. The method according to claim 2, wherein the voluntary skeletal muscle exercise module comprises a hand gripping exercise instruction to grip a fist of the subject at a rate matching a displayed animation on the electronic device.

7. The method according to claim 2, wherein the voluntary skeletal muscle exercise module comprises an arm shaking exercise instruction to move an object displayed on the electronic device across the screen with an index finger of the subject.

8. The method according to claim 2, wherein the voluntary skeletal muscle exercise module comprises a leg lifting exercise instruct to lift their legs as measured by a change in a background area captured by the electronic device.

9. The method according to claim 1, wherein the one or more first modules comprise the vagal nerve stimulation module, and the vagal nerve stimulation module comprises at least one instruction selected from the group consisting of sense stimulation instructions for sight, sound, touch, taste, and smell.

10. The method according to claim 1, wherein the one or more first modules comprise the aerobic exercise module, and the aerobic exercise module comprises one or more first instructions to increase adiponectin secretion in the subject.

11. The method according to claim 1, wherein the one or more first modules comprise the relaxation module, and the relaxation module comprises one or more first instructions to increase adiponectin secretion in the subject.

12. The method according to claim 1, wherein the subject is a late cancer patient, and the method excludes providing a voluntary skeletal muscle exercise module.

13. The method according to claim 1, wherein the sensor comprises one or more of a camera, an accelerometer, a magnetometer, a light sensor, a microphone, a proximity sensor, a touch sensor, a gyroscope, a Global Positioning System (GPS) sensor, an ambient light sensor, a fingerprint sensor, a pedometer, a heart rate sensor, and a thermometer.

14. The method according to claim 1, wherein the sensor comprises a touch sensor, and the subject provides the adherence information to the electronic device using the touch sensor.

15. A system for treating cancer cachexia in a subject, comprising:
a digital apparatus configured to execute a digital application comprising one or more first modules, for treating cancer cachexia in a subject, wherein the digital apparatus comprises a sensor for sensing adherence by the subject to a first set of instructions of the one or more first modules;
a healthcare provider portal configured to provide one or more options to a healthcare provider to perform one or more tasks to prescribe treatment for the cancer cachexia in the subject based on information received from the digital application; and
an administrative portal configured to provide one or more options to an administrator of the system to perform one or more tasks to manage access to the system by the healthcare provider.

16. The system according to claim 15, wherein the digital apparatus generates digital therapeutic modules based on biochemical factors related to the cancer cachexia.

17. The system according to claim 15, wherein the digital application transmits data to a server, and wherein the server receives one or more second instructions from an external reviewer.

18. The system according to claim 15, wherein the one or more options provided to the healthcare provider are selected from the group consisting of adding or removing the subject, viewing or editing personal information for the subject, viewing adherence information for the subject, viewing a result of the subject for one or more at least partially completed digital therapeutic modules, prescribing one or more digital therapeutic modules to the subject, altering a prescription for one or more digital therapeutic modules, and communicating with the subject.

19. The system according to claim 15, wherein the one or more options comprise the viewing the adherence information, and the adherence information of the subject comprises one or more of a number of scheduled or prescribed digital therapeutic modules completed by the subject, and a calendar identifying one or more days on which the subject completed, partially completed, or did not complete one or more scheduled or prescribed digital therapeutic modules.

20. The system according to claim 15, wherein information, which is received from the subject, comprises at least one of basal factors, medical information, or digital therapeutics literacy of the subject,
the basal factors including the subject's activity, heart rate, sleep, and diet which includes nutrition and calories,
the medical information including the subject's electronic medical record (EMR), family history, genetic vulnerability, and genetic susceptibility, and
the digital therapeutics literacy including the subject's accessibility, and technology adoption to the digital therapeutics and the apparatus.

21. The system according to claim 15, wherein the outcome collection unit collects execution outcomes of digital instructions by monitoring the subject's adherence to the digital instructions or allowing the subject to directly input the subject's adherence to the digital instructions.

22. A computing system for treating cancer cachexia in a subject in need thereof, comprising:
a display configured to provide, to the subject, one or more first modules selected from the group consisting of a voluntary skeletal muscle exercise module, a vagal nerve stimulation module, an aerobic exercise module, and a relaxation module, each of the one or more first modules comprising one or more first instructions for the subject to follow;
a sensor configured to sense adherence by the subject to the instructions of the one or more first modules;
a transmitter configured to transmit adherence information, based on the adherence, to a server; and
a receiver configured to receive, from the server, one or more second instructions based on the adherence information,
wherein the display is further configured to provide, to the subject, one or more second modules selected from the group consisting of a voluntary skeletal muscle exercise module, a vagal nerve stimulation module, an aerobic exercise module, and a relaxation module, each of the one or more second modules comprising the one or more second instructions.

23. A non-transitory computer readable medium having stored thereon software instructions for treating cancer cachexia in a subject in need thereof that, when executed by a processor, cause the processor to:
display, by an electronic device to the subject, one or more first modules selected from the group consisting of a voluntary skeletal muscle exercise module, a vagal nerve stimulation module, an aerobic exercise module, and a relaxation module, each of the one or more first modules comprising instructions for the subject to follow;
sense, by a sensor in the electronic device, adherence by the subject to the instructions of the one or more first modules;
transmit, by the electronic device, adherence information, based on the adherence, to a server;
receive, from the server, one or more second instructions based on the adherence information; and
display, to the subject, one or more second modules selected from the group consisting of a voluntary skeletal muscle exercise module, a vagal nerve stimulation module, an aerobic exercise module, and a relaxation module, the one or more second modules comprising the one or more second instructions.
